(19) United States
(12) Patent Application Publication
(10) Pub. No.: US 2026/0297138 A1
(43) Pub. Date: Oct. 1, 2026

(54) AAV CAPSIDS FOR ON-TARGET DELIVERY TO SPINAL CORD

(71) Applicant: The General Hospital Corporation, Boston, MA (US)

(72) Inventors: Casey A. Maguire, Arlington, MA (US); Killian S. Hanlon, Boston, MA (US); Demitri Alexander de la Cruz, Boston, MA (US); Mingjie Cheng, Boston, MA (US); Miguel Israel Chavez Santoscoy, Boston, MA (US)

(21) Appl. No.: 19/476,019

(22) PCT Filed: Apr. 17, 2024
(86) PCT No.: PCT/US2024/024996

§ 371 Date: Oct. 16, 2025

Related U.S. Application Data
(60) Provisional application No. 63/460,008, filed on Apr. 17, 2023.
(60) Provisional application No. 63/581,492, filed on Sep. 8, 2023.

(51) Int. Cl. C07K 14/005; C12N 15/86

(52) U.S. Cl. CPC C07K 14/005; C12N 15/86; C12N 2750/14122; C12N 2750/14143

(57) ABSTRACT

Provided herein are engineered AAV capsids with improved biodistribution and transduction efficiency in the spinal cord and lowered biodistribution to liver, compositions comprising the capsids, and methods of using the same.

### CLAIM OF PRIORITY

This application claims the benefit of U.S. Provisional Patent Application Ser. No. 63/460,008, filed on Apr. 17, 2023, and 63/581,492, filed on Sep. 8, 2023. The entire contents of the foregoing are hereby incorporated by reference.

### FEDERALLY SPONSORED RESEARCH OR DEVELOPMENT

This invention was made with Government support under Grant No. DC017117 awarded by the National Institutes of Health. The Government has certain rights in the invention.

### SEQUENCE LISTING

This application contains a Sequence Listing that has been submitted electronically as an XML file named 29539-0747WO1_SL_ST26.xml. The XML file, created on Apr. 16, 2024, is 59,108 bytes in size. The material in the XML file is hereby incorporated by reference in its entirety.

### TECHNICAL FIELD

Provided herein are engineered AAV capsids with improved biodistribution and transduction efficiency in the spinal cord and lowered biodistribution to liver, compositions comprising the capsids, and methods of using the same.

### BACKGROUND

Genetic diseases that affect the spinal cord are devastating, owing to their debilitating effects on quality of life as well as lack of effective treatments. Gene therapy for the central nervous system (CNS) using adeno-associated virus (AAV) vectors has risen to the forefront with promising clinical data showing efficacy of therapy targeting the CNS and spinal cord^{1, 2}. Many of these approaches use systemic delivery of AAV serotypes such as AAV9 which can cross the blood-brain barrier. This generally gives reasonable distribution of transgene expression in neurons throughout the brain and spinal cord, although the efficiency with AAV9 and natural serotypes is fairly low and requires high doses of vector³. Systemic dosing also has major translational challenges such as pre-existing neutralizing antibodies to the AAV capsid, the high cost of generating the massive doses (>10¹⁴ vg/kg) required for therapy, and even dose limiting toxicities due to apparent complement activation and liver dysfunction^{4, 5}. There is also concern of off target expression of transgene in dorsal root ganglion (DRG) neurons causing toxicity, as has been demonstrated in large animal models⁶, although this can be mitigated with miRNA transgene expression detargeting strategies⁷.

### SUMMARY

Described herein are engineered capsids, including AAV capsid proteins comprising an amino acid sequence that comprises at least four, at least five, at least six, or all seven contiguous amino acids from the sequence TR2 (RTTASLM, SEQ ID NO:12), NL1 (LTTEGRR, SEQ ID NO:10), TH1 (HPARALP, SEQ ID NO:13), or TP1 (PKYPLLG, SEQ ID NO:14), or another amino acid sequence described herein, e.g., in any of Tables III, IV, V, VIA, VIB, or VIC.

In some embodiments, the AAV is AAV9, e.g., AAV9 VP1. In some embodiments, the sequence is inserted in a position corresponding to amino acids 588 and 589 of AAV9 VP1.

Also provided herein are nucleic acids encoding the AAV capsid proteins described herein, optionally as recombinant episomes or viral vectors.

Further, provided herein are AAV comprising a capsid protein as described herein, and preferably not comprising a wild type VP1, VP2, or VP3 capsid protein.

In some embodiments, the AAV further comprises a transgene, preferably a therapeutic transgene, e.g., a protein coding sequence or inhibitory nucleic acid as listed in Table A.

Additionally, provided herein are methods for delivering a transgene to a cell. The methods comprise contacting the cell with an AAV comprising a capsid protein as described herein. In some embodiments, the cell is a neuron (optionally a dorsal root ganglion neuron or spiral ganglion neuron), astrocyte, glial cell, Schwann cell of a peripheral nerve, or cardiomyocyte. In some embodiments, the cell is in a living subject, e.g., a mammalian subject. In some embodiments, the cell is in a tissue selected from the brain, spinal cord, dorsal root ganglion, Schwann cell of a peripheral nerve, or heart, and a combination thereof. In some embodiments, the subject has a disease listed in Table A, and the methods include delivering an AAV comprising a capsid protein as described herein and a protein coding sequence or inhibitory nucleic acid as listed in Table A.

In some embodiments, the cell is in the spinal cord of the subject, and the AAV is administered by intrathecal delivery. In some embodiments, the intrathecal delivery is via lumbar injection, cisternal magna injection, or intraparenchymal injection.

In some embodiments, the AAV is delivered by parenteral delivery, preferably via intravenous, intraarterial, subcutaneous, intraperitoneal, or intramuscular delivery.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Overview of the in vivo selection in non-human primates (NHPs) to identify AAV capsids with the ability to efficiently transduce spinal cord. a. Round 1 of selection. The iTransduce AAV genome plasmid has a CBA-driven Cre cassette followed by a p41 driven AAV9 capsid with randomized 21mer bp inserts encoding for 7mer peptides inserted into the capsid. NHPs are injected intrathecally (IT) with the library and three weeks later animals are sacrificed and spinal cord removed. Next generation sequencing (NGS) is performed on AAV genomes containing 21mer inserts amplified from three spinal cord regions. The rescued cap inserts are inserted into the iTransduce backbone and packaged into the RI rescued library. b. Round 2 of selection. A second reporter AAV expression plasmid is used to allow transduction-based selection in a non-transgenic animal. The reporter contains a Cre-sensitive floxed-STOP-H2B-mPlum cassette. When co-injected intrathecally into NHPs with iTransduce library from Round 1, cells that are co-transduced with Cre-expressing capsids can rescue nuclear mPlum expression. Spinal cord is again isolated and DNA isolated using three different strategies: (1) whole tissue DNA as in 1a, (2) flow-sorted nuclei, (3) flow-sorted H2B-mPlum nuclei (contains transduction-competent capsid DNA). NGS is performed and AAV capsids are identified for further screening for spinal cord transduction.

FIGS. 2A-C. Two-vector iTransduce system allows detection of transduction competent AAV in vitro and in vivo. a. 293T cells were transduced with AAV-Cre or AAV-Floxed-STOP-H2B-mPlum vectors alone (first two columns) or co-transduced with both vectors (far right column). Nuclear H2B-mPlum fluorescence was only detected in co-transduced cells. Scale bar=100 μm. b. Wild type C57Bl/6 mice were iv injected with the following vector groups (n=2 mice/group): (1) AAV-Floxed-STOP-H2B-mPlum, (2) AAV-Cre, or (3) a mixture of both vectors in 1 and 2. c. Brains were harvested, frozen, and nuclei isolated and then labeled with violet dye and analyzed with flow cytometry for violet and H2B-mPlum.

FIGS. 3A-E. Isolation and selection of candidate variants in NHPs. a-b. Flow cytometry of nuclei isolated from control (a) and library-injected (b) NHPs. Nuclei were selected using fluorescent dye, with mPlum used to discriminate nuclei from transduced cells. c. Candidate variants were selected from whole tissue isolation, mPlum− nuclei, and mPlum+ (transduced) nuclei. The names and insert peptides of each variant are listed, corresponding to SEQ ID NOs:8, 4, 5, 7, 6, 9, 10, 11, 16, 14, 15, and 12. d. The frequency of amino acids at each position for the selected candidates is given. Larger size indicates more frequent incorporation of a given amino acid, for a given position. e. Overall production yield of each capsid variant when individually produced, compared to AAV9 (in black).

FIGS. 4A-G. Barcoded candidate capsid screen identifies variants with enhanced biodistribution in NHP spinal cord and reduced biodistribution to liver. A. Schematic showing the procedure used for this experiment. Each capsid variant was barcoded between the protein-coding region and post-transcriptional sequences. Following intrathecal injection, DNA was isolated from 1) each region of the spinal cord, 2) liver, 3) brain, 4) peripheral nerves, and 5) heart. These were subjected to next-generation sequencing. b. Overall biodistribution of AAV capsid genomes in each section of the spinal cord as measured by qPCR. L1, T1, C1 are from NHP #1001. L2, T2, C2 are from NHP #1002. C. Heatmap showing relative frequency of each variant (or AAV9) in each region of the spinal cord and liver for each animal. Variants are clustered by expression pattern (higher expression=lighter color). d-e. Average spinal cord (d) and liver (e) biodistribution of each variant expressed as fold-change vs. AAV9. Zero values are not displayed owing to logarithmic scaling of the graphs. Error bars represent standard error. L, T, C (L1=NHP #1001, L2=NHP #1002, etc.): Lumbar, Thoracic, Cervical regions of the spinal cord. Li (Li1, 2)=Liver samples for #1001 and #1002, respectively. f. Biodistribution of AAV variants in different regions of spinal cord. Data is shown from lumbar (top graph), thoracic (middle graph) and cervical (bottom graph) regions of spinal cord. Zero values are not displayed owing to logarithmic scaling of graphs. Error bars represent standard error. Figure is a separated version of FIG. 4d. g. Biodistribution of AAV variants in the heart after spinal cord delivery. Zero values are not displayed owing to logarithmic scaling of graphs. Error bars represent standard error.

FIGS. 5A-C. Barcoded capsid screen identifies variants with enhanced transgene RNA levels in spinal cord and reduced RNA levels in liver. a. Expression levels of the AAV transgene in the lumbar portion of the spinal cord of each NHP (#1001, #1002), relative to GAPDH mRNA. b-c. Average RNA expression values for the lumbar region of the spinal cord (b) and liver (c), expressed as fold-change vs. AAV9. Zero values are not displayed owing to logarithmic scaling of the graphs. Error bars denote standard error. Lumbar 1=NHP #1001; Lumbar 2=NHP #1002. Liver 1=NHP #1001; Liver 2=NHP 1002.

FIGS. 6A-F. NHP selected AAV capsid candidates transduce neurons and glia in mice after lumbar intrathecal bolus injection. a. Mice were injected intrathecally with AAV9, NL1, TH1, TP1, and TR2 packaging an AAV-CBA-GFP transgene cassette. All capsids were injected at 8.3×10¹⁰ vg/mouse with the exception of NL1, which was dosed at 4.8×10¹⁰ vg/mouse. b. Analysis of GFP expression in spinal cords by ELISA (n=4-5 mice/capsid). Each data point represents a spinal cord segment (either rostral or caudal) from an individual mouse. Error bars represent standard deviation of the mean. ns=not significant; *p<0.02. c. Representative low and high magnification images of spinal cord for each capsid. Brown DAB (3,3′-Diaminobenzidine) staining indicates GFP immunoreactivity. Black arrowheads point to cells with neuronal morphology. Cyan arrowhead indicates a cell with glial morphology. d. Score grading for spinal cord transduction efficiency. e. Transduction scoring for neurons in spinal cord. f. Transduction scoring for glial cells in spinal cord. Each data point represents either a rostral or caudal region for each mouse analyzed. e, f represent data from n=5 mice/capsid.

FIGS. 7A-B. Validation of barcoded AAV genomes to detect differences in biodistribution and transgene expression of validate capsids. a, An AAV expression plasmid was constructed with a CBA promoter, hFrataxin cDNA with a HA tag, and a DNA barcode that can be measured at the DNA and RNA levels. Unique barcodes were made for AAV9 and AAV-F and packaged into these capsids. Mice (n=2) received a 1:1 mixture of the capsids by tail vein injection, and liver and brain were harvested for measurement of barcode reads by NGS for both DNA and RNA. b, Quantitation of reads obtained in the liver and brain. Biological replicates are shown for each group. Only one mouse is shown for liver mRNA reads due to a contamination issue with one of the mouse livers. All other samples show two biological replicates. Error bars depict the standard deviation from the mean.

FIG. 8. Variant frequency in non-spinal cord tissues of NHPs injected intrathecally with barcoded AAV capsid candidates. Heatmap showing relative frequency of each AAV variant in various regions of the CNS, along with heart and liver. Variants are clustered by expression pattern. Number (1, 2) refers to each animal. Parietal, parietal lobe. Prefrontal, prefrontal cortex. DRG, dorsal root ganglion.

FIG. 9. Variant frequency in peripheral nerve tissues of NHPs injected intrathecally with barcoded AAV capsid candidates. Heatmap showing relative frequency of each AAV variant in peripheral nerves. Variants are clustered by expression pattern. Number (1, 2) refers to each NHP (#1001 and #1002). 8th, 8th cranial nerve. Sci, sciatic nerve. Su, sural nerve. Ul, ulnar nerve.

FIGS. 10A-C. Transduction of mouse liver by NHP selected AAV capsid candidates after lumbar intrathecal bolus injection. Mice (n=5/capsid) were injected intrathecally with AAV9, NL1, TH1, TP1, and TR2. All capsids were injected at 8.3×1010 vg/an with the exception of NL1, which was dosed at 4.8×1010 vg/an a. Sample image of transduced liver. Brown DAB (3,3′-Diaminobenzidine) staining indicates GFP immunoreactivity. b. scoring grading for transduction efficiency. c. Transduction scoring for various cell types in the mouse liver. Each data point represents one mouse.

FIGS. 11A-H. NHP selected capsids efficiently transduce human spinal cord organoids (SCOs). a. Schematic of experiment. A single-stranded AAV genome with a GFP expression cassette was packaged inside each of the top four candidate and AAV9 capsids and used to transduce human SCOs. b. Representative whole organoid images showing intrinsic GFP expression (day 6 post transduction) by each capsid in younger and older organoids. c. Quantitation of max-projection images of all organoids per group (n=4-6/capsid). Day 66 SCOs: *p=0.025; **p=0.006; ****p<0.0001. Day 225 SCOs: *p=0.025, ***p=0.0005, ****p<0.0001. d, e. Colocalization of GFP with markers for neurons and astrocytes in day 225 old SCOs. f. Schematic for quantitation of confocal z-stacks to quantitate transgene expression throughout the organoids. g. Fluorescence intensity taken at 35 μm intervals starting at the top of the imaging stack (280 μm total depth analyzed). h. GFP positive cell counts for each group taken from three slices near the top, middle, and bottom of the z-stack.

FIGS. 12A-E. Analysis of spinal cord organoids transduced with AAV variants (SCO laboratory 2). a. Quantification of mean GFP intensity in whole live organoids at days 1, 7, and 10. Day 7 GFP signal of NL1 and TR2 was increased in comparison to AAV9 (n=2; P=0.03 and 0.0059, respectively). Day 10 GFP signal of all AAV variants was superior to AAV9 (n=2; NL1 P<0.0001, TH1 P=0.049, TP1 P=0.0146, TR2 P<0.0001). Quantifications were performed in 3 organoids per hiPSC line per capsid. P values were calculated using 2way ANOVA with Bonferroni's multiple comparison correction test. b. Representative immunofluorescent confocal images of SCOs transduced with AAV9 and AAV variants stained against GFP in green and NeuN in red at day 55, 10 days post transduction (scale bar 100 m). c. Quantification of the percentage of GFP+ cells in transduced organoids 10 days post transduction (n=2). Quantifications were performed in 5 to 7 organoids between both hiPSC lines, with >5000 DAPI nuclei included. (*P<0.05; **P<0.01). P values were calculated using nested 1way ANOVA with Bonferroni's multiple comparison correction test. d. Quantification of AAV genomes in organoids 10 days post transduction (n=2). 3 organoids per hiPSC line per capsid were pulled to perform RNA isolation. (*P<0.05; **P<0.01). e. Quantification of the percentage of GFP+ISL1+ motor neurons in transduced monocultures 6 days post transduction. Quantifications were performed with 2× images per coverslip per hiPSC line per capsid; one coverslip per line, with >175 ILS1+ neurons included. No statistical significance was reach using nested 1way ANOVA with Bonferroni's multiple comparison correction test.

### DETAILED DESCRIPTION

Research has been focused on developing AAV-based gene therapies using direct injection of vector into the cerebral spinal fluid (CSF). Intrathecal injection into the cerebral spinal fluid (CSF) around the lumbar spinal cord with AAV generally gives good transduction of motor neurons in the lumbar region, but less transduction of cervical regions and the brain^{8, 9}. Positioning the subject in the Trendelenburg position can enhance vector biodistribution of the cervical region somewhat^{9, 10} On the other hand, cisterna magna injection of AAV vectors gives better cervical and brain transduction but less so in the lumbar region¹¹. There is also a surprisingly high amount of vector that enters the circulation after CSF delivery which ends up in the liver^{12, 13}, which could lower the safety benefits of a more local delivery. In fact a recent study by Hudry et al., the team demonstrated liver toxicity in cynomolgus monkeys after intrathecal delivery¹⁴. And in a clinical trial for spinal muscular atropy (SMA), 2 out of 25 patients receiving intrathecally administered AAV9-sc-CBA-SMN1 (onasemnogene abeparvovec) had liver enzyme elevations¹⁵. There is a clear need for AAV vectors that can transduce the spinal cord with the following properties: (1) high efficiency at lower dose, (2) distribution throughout the entire spinal cord from lumbar to cervical regions, (3) low off target leakage/transduction of peripheral organs, especially liver.

With this in mind, we developed an in vivo selection strategy with an AAV9 peptide display library (iTransduce) that allows selection of transduction competent AAV capsids¹⁶. We further developed the system to be utilized in non-human primates (NHPs) to maximize clinical translation of the capsids' transduction properties, owing to physiological and anatomical (e.g., size, CSF volume) differences between primates and mice. We performed two rounds of in vivo selection in NHPs after intrathecal injection of the AAV peptide display library. In the second round, we utilized iTransduce to isolate AAV variants that led to functional transduction. We then identified capsids that outperformed AAV9 in terms of biodistribution and transduction efficiency in the spinal cord and lowered biodistribution to liver, as well as enhanced transduction of human spinal cord organoids.

While AAV9 has been successfully developed for transgene delivery to the spinal cord to treat the neuromuscular disease, spinal muscular atrophy (SMA), there is room for improvement. When delivered at high systemic doses, AAV vectors can cause liver enzyme elevation, complement activation, thrombotic microangiopathy (TMA), and even sepsis, organ failure, and even death^{4, 19} Although immunosuppressive/modulatory regimens may mitigate some of these serious adverse events³², these pharmacological interventions are not without risk and may not be feasible/effective in all patients. Thus, much recent research is focused on developing AAV gene therapies for CNS disorders using a CSF route of administration.

Delivery of AAV directly into the CSF has been shown to reduce exposure to neutralizing antibodies which are present at much lower levels than in blood²⁰. While there is less systemic exposure to vector than a systemic route of administration, there is still a large amount of vector that can enter the blood and peripheral organs after CSF injection. A recent study by Meseck et al. performed lumbar IT injection of NHPs and measured vector genomes in CNS and peripheral tissues 4 weeks post injection¹². Remarkably, liver received over 100 times the number of AAV genomes compared to the brain and approximately 10 times more than the spinal cord after lumbar intrathecal injection¹². Furthermore, transgene expression was higher in the liver and heart compared to spinal cord¹². Other studies using radiolabeled AAV capsids injected into the CSF of NHPs—including AAV9—has demonstrated that around 50% of administered capsid can enter the periphery¹³. Obviously, this high off-target biodistribution runs completely counter to the intent of a direct CSF administration. Transduction of liver after intrathecally injected AAV is more than just a theoretical risk for toxicity. Recently Hudry et al. found that both intravenously and intrathecally injected AAV9 encoding a self-complementary GFP or SMN1 transgene cassette resulted in hepatotoxicity as measure by liver enzyme elevations and chemistry, histopathology, cytokines, and complement activation¹⁴. Immunosuppression with Prednisolone_ or Rituximab/Everolimus were not able to mitigate these effects, suggesting that current pharmaceuticals will not suffice¹⁴. Interestingly, the researchers found that AAV transduction needed to occur, as empty capsids and promoterless transgene cassettes did not result in toxicity¹⁴. The most straightforward way to avoid transgene expression in the liver is to either avoid biodistribution to liver using engineered capsids like the ones described in our study, or restrict transgene expression away from the liver using tissue specific promoters²¹ or miRNA based transgene mRNA degradation^{22, 23}.

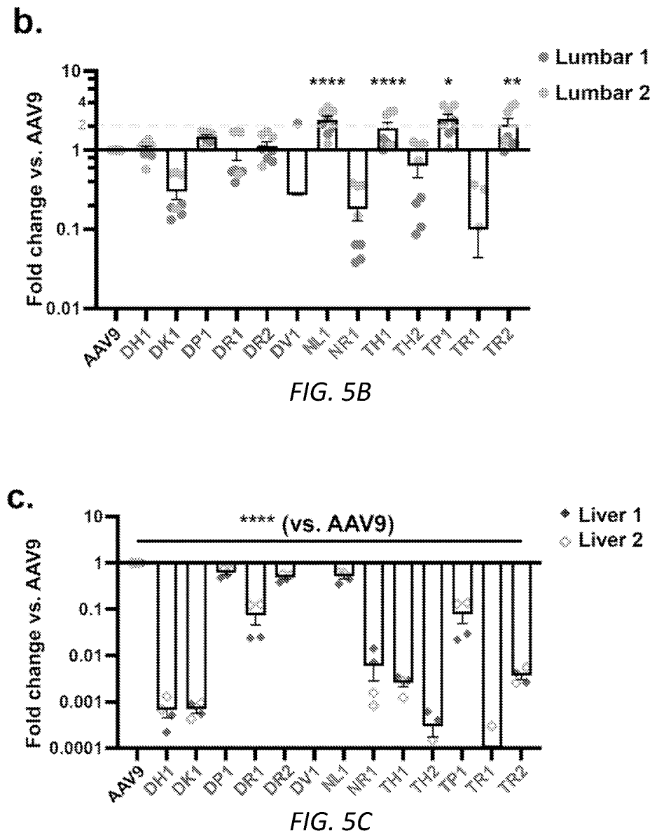

The present disclosure is, to the best of the inventors' knowledge, the first selection of an AAV peptide display library in NHPs using the intrathecal route. Remarkably, the majority of the top capsids identified in our selection showed greatly reduced biodistribution (up to 1,250-fold less frequent reads) and transduction (up to 30,000-fold less frequent reads) in liver compared to AAV9. This desirable feature may allow for less toxicity observed with liver transduction, although this will need to be confirmed by testing with individual capsids encoding transgenes of interest in toxicology studies in NHPs. Furthermore, the top candidate capsids appear to be more efficient than AAV9 at spinal cord transduction at the RNA level (FIG. 5b). This latter outcome may have been influenced by the selection approach. To enable detection of transgene expressing cells during the selection rounds, we made some modifications to our prior reported iTransduce system¹⁶ to allow its use in non-transgenic animals. To do this we used a two-vector system with the library encoding Cre recombinase as usual and the second vector packaging the Cre inducible reporter (Floxed-STOP-H2B-mPlum). Using this system, we were able to flow sort H2B-mPlum fluorescent nuclei that applied additional selective pressure to identify transduction-competent capsids. The reporter capsid (AAV9 in our case) likely influences the selection outcome, as fluorescent nuclei are restricted to cells which can be transduced by AAV9. In our study, this was a benefit, as we wanted to maintain the tropism of AAV9 in the spinal cord (glia and neurons), while improving transduction efficiency and biodistribution. However, if other cell types outside the tropism of AAV9 are desired as targets, this approach may not be feasible, or a different capsid packaging the reporter gene which can transduce the target cell would need to be utilized.

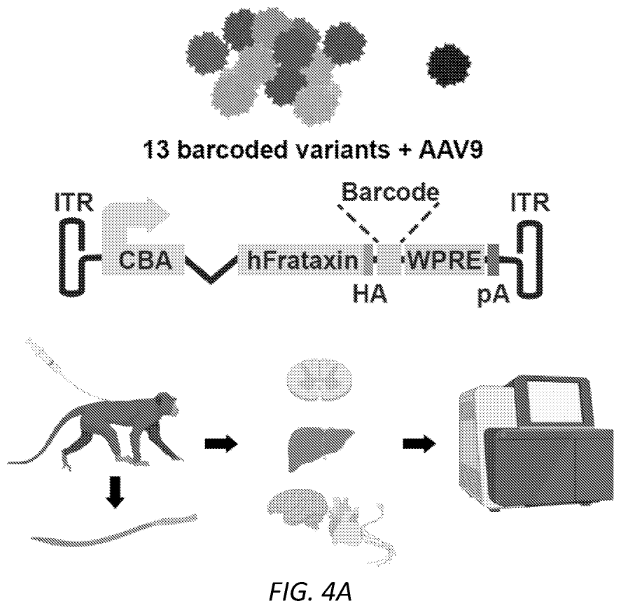

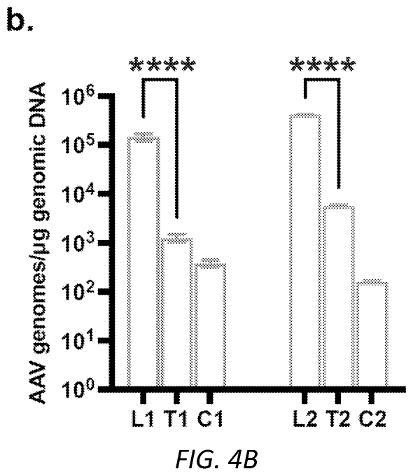

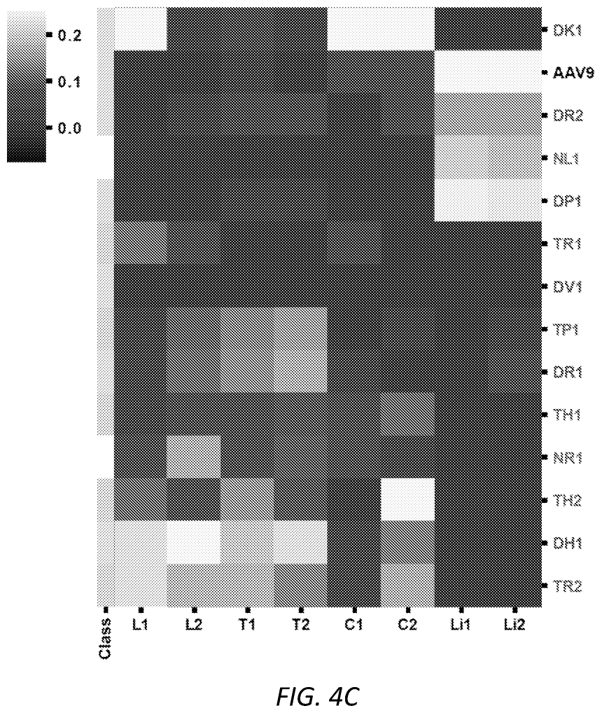

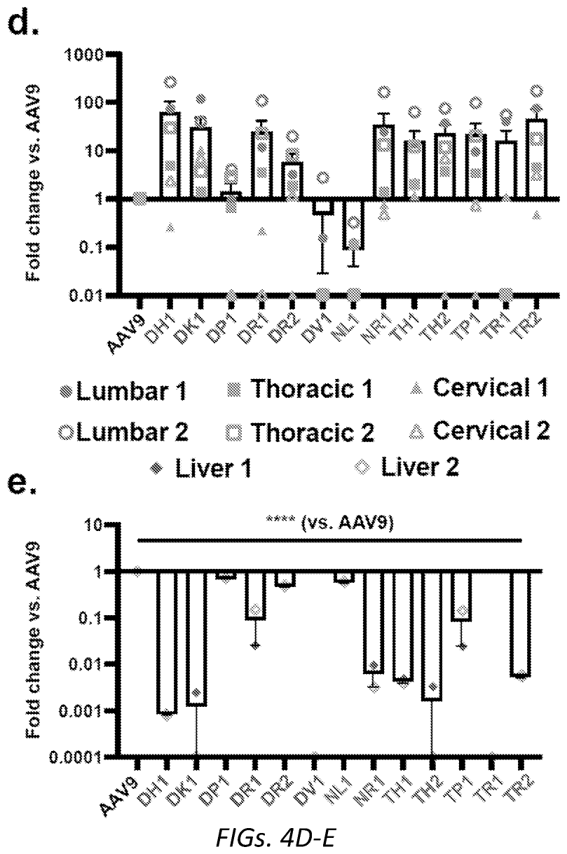

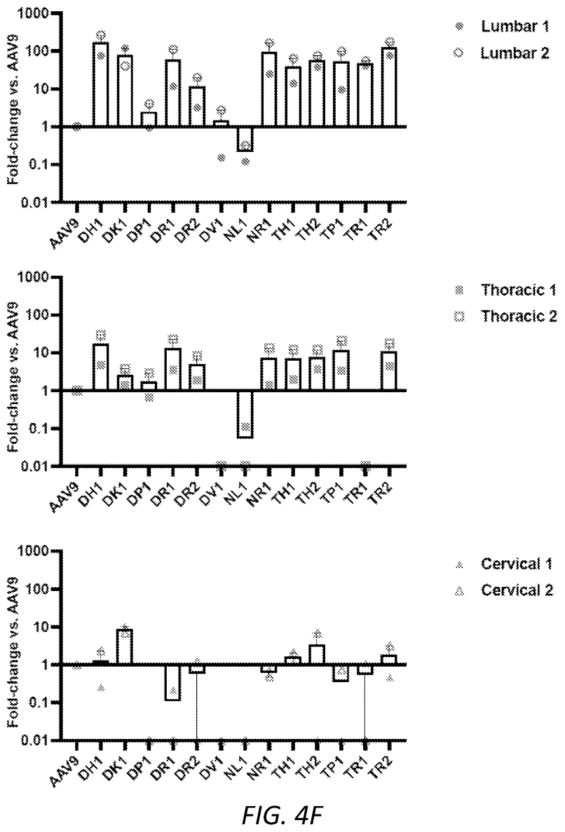

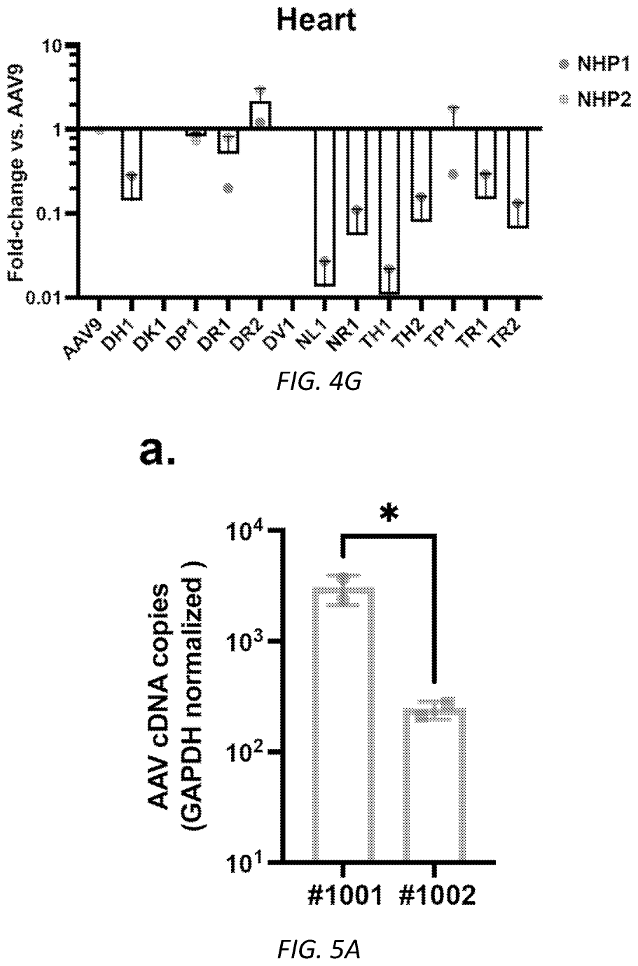

We made several interesting observations during the study. First, many of the peptides had a leucine:proline (LP) motif in the 7-mer sequence, which may be important in their enhanced biodistribution in the spinal cord compared to AAV9 (FIG. 4d). Second, we found that the capsids isolated from nuclei or from nuclei in transduced cells had higher levels of transduction (RNA-based reads) compared to capsids identified from the whole-tissue isolated DNA (FIG. 5b). Whole tissue isolation is likely to contain AAV genomes that may be outside the cell (still in capsids) or nucleus or not uncoated in the nucleus (not transcriptionally active). On the other hand, nuclear isolation and transgene-based selection is at or close to the final steps of transduction. While this observation was based on a limited number of capsids from each method, owing to a limit of capsids that we could test by barcoding, it does suggest, in line with other reports^{16, 24-26}, that selection as far down stream in the transduction process as technically feasible, is likely to yield capsids capable of transduction. While we did observe capsids that appear more efficient than AAV9 at both the biodistribution (DNA) and transduction (RNA) level, the fold differences for RNA were much lower than for DNA. This could be due to several reasons. First, because we did a pooled injection, the injected dose of vector was quite low (4.48×10¹² vg), with most capsids represented at only 4×10¹¹ vg each, so transgene RNA levels were quite low in spinal cord (see RT-qPCR, FIG. 5a) which could only be reliably detected in the lumbar region. We couldn't assess the transduction in the thoracic or cervical regions. This dose of the pooled library is approximately 3 to 10-fold lower than what is typically delivered for therapeutic intrathecal dosing studies in NHPs and 30 to 100-fold lower at the individual capsid level. Individual testing of the top performing capsids and AAV9 in NHPs entails long wait times owing to limited availability of animals. Furthermore, it is ethically questionable and cost prohibitive in most academic labs to test multiple capsids in the number of NHPs required to achieve statistically relevant comparisons. Human organoid systems are being pursued as preclinical models for drug and gene therapy testing, including AAV vectors³³⁻³⁶. Here, we used spinal cord organoids to assess the cellular tropism and transduction efficiency/potency of our selected candidates compared to AAV9. We observed that our selected capsids had higher transgene expression efficiency (in both the number of cells transduced and the level of transgene product, e.g. GFP intensity) compared to AAV9. We also found that the candidates could transduce motor neurons and glia, two of the major clinically relevant cell types for spinal cord gene therapy. These data should be impactful for the field as they demonstrate that capsids selected with desired properties in NHPs were effective in a 3D human organoid preclinical model. The human organoid model could serve important roles in preclinical development of gene therapy. First, they could help narrow down the list of candidates from initial pooled barcoded AAV's such as we did. The human spinal cord organoid data helped us to narrow our top candidates to TR2 being the lead for further expensive NHP testing. Additionally, the organoids could serve in parallel to study a given therapies therapeutic effect if they contain disease-specific mutations (a feature the NHP model does not provide). Overall, for the work described here, the human SCO served to validate the capsids which we had chosen from our NHP selections were potent at transduction of clinically relevant cells. Furthermore, these new capsids should serve as very useful gene delivery tools for basic biology studies in spinal cord organoids.

We tested the top chosen candidates for transduction of murine spinal cord after intrathecal injection to see if they would be functional in the most common preclinical model of human disease, mice. Using immunohistochemical analysis of GFP-expressing cells to indicate spinal cord transduction, we found that these capsids, like AAV9, transduce clinically relevant cell types such as neurons and glia. Interestingly, capsids TH1 and TR2 yielded statistically significant higher transduction (GFP levels) than AAV9 and thus these capsids should be useful in preclinical gene therapy studies in mice via the intrathecal route.

While the NGS reads coming from RNA provided evidence that many of the selected capsids were more functional at transduction of spinal cord compared AAV9, we could not determine which cell types were transduced (anti-HA Frataxin staining of spinal cord did not show any signal, data not shown). Thus, to understand if the capsids could transduce main targets of gene therapy in the CNS, astrocytes and neurons, we used intrathecal lumbar injection of mice as a surrogate. This experiment also tested whether the NHP-selected capsid transduction would occur in the most common preclinical models of human disease, mice. Using a semi-quantitative analysis of spinal cord transduction, we found that these capsids, like AAV9, transduced clinically relevant cell types such as neurons and glia. In most cases, transduction efficiency in mice was similar to AAV9, so these capsids may be used for pre-clinical work in mice.

While not a primary objective of the study, we also assessed AAV encoded DNA barcode frequency for the pooled capsids in brain and peripheral nerves. From these data, certain capsids appear to outperform AAV9 in biodistribution to certain brain regions and nerves. This includes capsid DH1 in brain and DH1, DP1, and DR2 in nerves. It may be of future interest to test these individual capsids in NHPs after intrathecal injection to assess transduction of brain and nerves.

Thus, described herein are capsids with enhanced biodistribution and transduction of spinal cord in NHPs compared to AAV9. Coupled with the lower liver biodistribution and transduction, the capsids described here, including NL1 (LTTEGRR, SEQ ID NO:10), TH1 (HPARALP, SEQ ID NO:13), TP1 (PKYPLLG, SEQ ID NO:14), and TR2 (RTTASLM, SEQ ID NO:12), as well as other capsids described herein, e.g., listed in Table III, can be used for gene delivery in subjects with spinal cord injury and disease.

### Engineered AAV Capsid Proteins

The present methods identified peptide sequences that enhance biodistribution and transduction of spinal cord of an AAV when inserted into the capsid of the AAV, e.g., into AAV1, AAV2, AAV8, or AAV9, or another AAV known in the art or listed herein. In some embodiments, the peptides comprise sequences of at least 7 amino acids. In some embodiments, the amino acid sequence comprises at least 4, e.g., 5, 6, or 7 contiguous amino acids of the sequences NL1 (LTTEGRR, SEQ ID NO:10), TH1 (HPARALP, SEQ ID NO:13), TP1 (PKYPLLG, SEQ ID NO:14), and TR2 (RTTASLM, SEQ ID NO:12), as well as other capsids listed herein, e.g., in any of Tables III, IV, V, VIA, VIB, or VIC.

Peptides including reversed sequences can also be used.

### AAVs

Viral vectors for use in the present methods, kits and compositions include recombinant adeno-associated virus (AAV) comprising a capsid peptide as described herein and optionally a transgene for expression in a target tissue (e.g., in an expression construct).

AAV are a preferred viral vector system for delivery of nucleic acids. AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV3, AAV4, AAV5, AAV6, AV6.2, AAV7, AAV8, rh.8, AAV9, rh.10, rh.39, rh.43 or CSp3; for CNS use, in some embodiments the AAV is AAV1, AAV2, AAV4, AAV5, AAV6, AAV8, or AAV9. As one example, AAV9 has been shown to somewhat efficiently cross the blood-brain barrier. Using the present methods, the AAV capsid can be genetically engineered to enhance biodistribution and transduction of spinal cord, by insertion of a peptide sequence as described herein into the capsid protein, e.g., into the AAV9 capsid protein VP1 between amino acids 588 and 589.

An exemplary wild type AAV9 capsid protein VP1 (Q6JC40-1) sequence is as follows:

```
(SEQ ID NO: 1)
                10 20 30 40
MAADGYLPDW LEDNLSEGIR EWWALKPGAP QPKANQQHQD
                50 60 70 80
NARGLVLPGY KYLGPGNGLD KGEPVNAADA AALEHDKAYD
                90 100 110 120
QQLKAGDNPY LKYNHADAEF QERLKEDTSF GGNLGRAVFQ
              130 140 150 160
AKKRLLEPLG LVEEAAKTAP GKKRPVEQSP QEPDSSAGIG
              170 180 190 200
KSGAQPAKKR LNFGQTGDTE SVPDPQPIGE PPAAPSGVGS
              210 220 230 240
LTMASGGGAP VADNNEGADG VGSSSGNWHC DSQWLGDRVI
              250 260 270 280
TTSTRTWALP TYNNHLYKQI SNSTSGGSSN DNAYFGYSTP
              290 300 310 320
WGYFDENRFH CHFSPRDWQR LINNNWGFRP KRLNFKLFNI
              330 340 350 360
QVKEVTDNNG VKTIANNLTS TVQVFTDSDY QLPYVLGSAH
              370 380 390 400
EGCLPPFPAD VEMIPQYGYL TLNDGSQAVG RSSFYCLEYF
              410 420 430 440
PSQMLRTGNN FQFSYEFENV PFHSSYAHSQ SLDRLMNPLI
              450 460 470 480
DQYLYYLSKT INGSGQNQQT LKFSVAGPSN MAVQGRNYIP
              490 500 510 520
GPSYRQQRVS TTVTQNNNSE FAWPGASSWA LNGRNSLMNP
              530 540 550 560
GPAMASHKEG EDRFFPLSGS LIFGKQGTGR DNVDADKVMI
              570 580 590 600
TNEEEIKTTN PVATESYGQV ATNHOSAQAQ AQTGWVQNQG
              610 620 630 640
ILPGMVWQDR DVYLQGPIWA KIPHTDGNFH PSPLMGGFGM
              650 660 670 680
KHPPPQILIK NTPVPADPPT AFNKDKLNSF ITQYSTGQVS
              690 700 710 720
VEIEWELQKE NSKRWNPEIQ YTSNYYKSNN VEFAVNTEGV
              730
YSEPRPIGTR YLTRNL
```

Thus provided herein are AAV that include one or more of the peptide sequences described herein, e.g., an AAV comprising a capsid protein comprising a sequence described herein, e.g., an AAV9 VP1 capsid protein wherein a peptide sequence described herein has been inserted into the sequence, e.g., between amino acids 588 and 589 (in bold above). The AAV can be, e.g., recombinant episomal AAV.

The AAV sequences can be, e.g., at least 80, 85, 90, 95, 97, or 99% identical to a reference AAV sequence set forth herein, e.g., can include variants, preferable that do not reduce the ability of the AAV to mediate transgene expression in a cell. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### Transgenes

In some embodiments, the AAV also includes a transgene sequence (i.e., a heterologous sequence), e.g., a transgene encoding a therapeutic agent, e.g., as described herein or as known in the art, or a reporter protein, e.g., a fluorescent protein, an enzyme that catalyzes a reaction yielding a detectable product, or a cell surface antigen. The transgene is preferably linked to sequences that promote/drive expression of the transgene in the target tissue, e.g., in an expression cassette (also referred to herein as an expression construct).

Exemplary transgenes for use as therapeutics include neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decarboxylase (AADC), aspartoacylase (ASPA), blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble gamma/delta T cell receptors, ligand-binding fragments of a soluble receptor, and the like; enzymes, such as α-glucosidase, imiglucarase, and β-glucocerebrosidase; enzyme activators, such as tissue plasminogen activator; chemokines, such as IP-10, monokine induced by interferon-gamma (Mig), Groa/IL-8, RANTES, MIP-1α, MIP-1β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2, and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other examples of protein of interest include ciliary neurotrophic factor (CNTF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, O-enolase, and glycogen synthase; lysosomal enzymes (e.g., beta-N-acetylhexosaminidase A); and any variants thereof.

The transgene can also encode an antibody, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2); TIGIT (T-cell Immunoreceptor with Ig and ITIM domains); B7-H3 (CD276); VSIR (V-set immunoregulatory receptor, aka VISTA, B7H5, C10orf54); BTLA 30 (B- and T-Lymphocyte Attenuator, CD272); GARP (Glycoprotein A Repetitions; Predominant; PVRIG (PVR related immunoglobulin domain containing); or VTCN1 (Vset domain containing T cell activation inhibitor 1, aka B7-H4).

Other transgenes can include small or inhibitory nucleic acids that alter/reduce expression of a target gene, e.g., siRNA, shRNA, miRNA, antisense oligos, or long non-coding RNAs that alter gene expression (see, e.g., WO2012087983 and US20140142160).

Other transgenes can include genome editing reagents including CRISPR proteins such as CRISPR-Cas9, -Cas12a nucleases and nickases, cytosine base editors (CBEs), adenine base editors (ABEs), CRISPR prime editors (PEs), variants thereof, and optionally their associated guide RNAs.

The expression cassette can also include one or more sequences that promote expression of a transgene, e.g., one or more promoter sequences; enhancer sequences, e.g., 5′ untranslated region (UTR) or a 3′ UTR; a polyadenylation site; and/or insulator sequences. In some embodiments, the promoter is a brain tissue specific promoter, e.g., a neuron-specific or glia-specific promoter. In certain embodiments, the promoter is a promoter of a gene selected from: neuronal nuclei (NeuN), glial fibrillary acidic protein (GFAP), MeCP2, adenomatous polyposis coli (APC), ionized calcium-binding adapter molecule 1 (Iba-1), synapsin I (SYN), calcium/calmodulin-dependent protein kinase II, tubulin alpha I, neuron-specific enolase and platelet-derived growth factor beta chain. In some embodiments, the promoter is a pan-cell type promoter, e.g., cytomegalovirus (CMV), beta glucuronidase, (GUSB), ubiquitin C (UBC), or rous sarcoma virus (RSV) promoter. The woodchuck hepatitis virus posttranscriptional response element (WPRE) can also be used.

microRNA (miRNA)-dependent post-transcriptional suppression of transgene expression can be used to increase specificity of vector-mediated transgene expression. MicroRNAs typically regulate gene expression by binding to sequences in the 3′ untranslated region (UTR) of the mRNA. To control exogenous transgene expression, tandem repeats of artificial microRNA target sites can be incorporated into the 3′ UTR of the transgene expression cassette, leading to subsequent degradation of transgene mRNA in cells expressing the corresponding microRNA, thereby decreasing expression. See, e.g., Geisler and Fechner, World J Exp Med. 2016 May 20; 6(2):37-54.

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078); or the addition of other peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience) 1-9, available at sfn.org/~/media/SfN/Documents/Short%20Courses/2011%20Short%20Course%20I/2011_SC1_Gray.ashx.

### Methods of Use

The methods and compositions described herein can be used to deliver any composition, e.g., a deoxyribonucleic acid sequence of interest to a tissue, e.g., to the spinal cord, and central nervous system (brain), heart, muscle, peripheral nervous system (e.g., dorsal root ganglion or peripheral nerves). In some embodiments, the methods include delivery to specific brain regions, e.g., cortex, cerebellum, hippocampus, substantia nigra, amygdala. In some embodiments, the methods include intrathecal delivery. In some embodiments, the methods include delivery to neurons, astrocytes, or glial cells in the spinal cord or Schwann Cells in the peripheral nerves.

In some embodiments, the methods and compositions, e.g., AAVs, are used to deliver a nucleic acid sequence to a subject who has a disease, e.g., a disease of the CNS; see, e.g., U.S. Pat. Nos. 9,102,949; 9,585,971; and US20170166926. In some embodiments, the subject has a condition listed in Table A; in some embodiments, the vectors are used to deliver a therapeutic agent (e.g., sequence encoding the target (gene addition therapy) or inhibitory nucleic acid that reduces expression of the target) listed in Table A for treating the corresponding disease listed in Table A. The therapeutic agent can be delivered as a nucleic acid, e.g., via a viral vector, wherein the nucleic acid encodes a therapeutic protein or an inhibitory nucleic acid such as an antisense oligo, siRNA, shRNA, or artificial miRNA that reduces expression of the target, and so on; or as a fusion protein/complex with a peptide as described herein.

The methods and compositions described herein can be used to treat these conditions in a subject in need thereof, by administration of a therapeutically effective amount of an AAV carrying a therapeutic transgene, sufficient to ameliorate, reduce risk of, or delay onset of one or more symptoms of the condition.

TABLE A
CNS and peripheral nerve targets

| | Target | | Target | |
|---|---|---|---|---|
| Disease | genes | Modality | cells/tissues | Reference |
| Alzheimer's Disease | CD33, | CD33: inhibitory | Brain | Griciuc et al., |
| | APOE, | nucleic acid | | 2013; Griciuc et |
| | BACE, | APOE: gene | | al., 2020 |
| | TREM2 | addition therapy | | |
| | | BACE: inhibitory | | |
| | | nucleic acid | | |
| | | against base or | | |
| | | encoded anti- | | |
| | | BACE1 antibodies. | | |
| | | TREM2: gene | | |
| | | addition therapy | | |
| Parkinson's Disease | GDNF, | Gene addition | Brain | Christine et al., |
| | AADC | therapy | Neurons | 2019 |
| X-linked | ABCD1 | Gene addition | Brain/spinal cord | Eichler et al., |
| Adrenoleukodystrophy/ | | therapy | Neurons/astrocytes/ | 2017; Gong et al., |
| Adrenomyeloneuropathy | | | microglia/ | 2015 |
| | | | endothelial | |
| | | | cells | |
| Canavan's Disease | ASPA | Gene addition | Brain | Leone et al., 2012 |
| | | therapy | | |
| Niemann Pick | NPC1 | Gene addition | Brain | Hughes et al., |
| | | therapy | | 2018 |
| Spinal muscular | SMN1, | Gene addition | Motor neurons | Al-Zaidy et al., |
| atrophy | SMN2 | therapy, or gene | | 2019 |
| | | editing of SMN2 to | | |
| | | convert to SMN1. | | |
| Huntington's | HTT | inhibitory nucleic | Brain | Caron et al., 2020; |
| Disease | | acid | neurons | Keskin et al., 2019 |
| Amyotrophic | SOD1, | SOD1: inhibitory | Spinal Cord/Motor | Mueller et al. |
| Lateral Sclerosis | SPTLC1, | nucleic acid | neurons | 2020 |
| (ALS) | C9ORF72 | SPTLC1: inhibitory | | |
| | | nucleic acid | | |
| | | knockdown of | | |
| | | pathogenic | | |
| | | mutations. Gene | | |
| | | editing | | |
| | | (correction) of | | |
| | | mutations. | | |
| | | C9ORF72: CRISPR | | |
| | | targeting of toxic | | |
| | | RNA | | |
| TSC1/TSC2 | Hamartin/ | Gene addition | Brain | Prabhakar et al. |
| | tuberin | therapy | | 2019; Cheah et al. |
| | | | | 2021 |
| Neurofibromatosis | NF2 | Gene addition | Peripheral nerves, | Prabhakar et al. |
| type 2 | (merlin) | therapy | vestibulocochlear | 2022 |
| | | | nerve | |
| Charcot Marie | GJB1, | Gene addition | Schwann | Kagiava et al. |
| Tooth Disease | PMP22. | therapy | cells/peripheral | 2021 |
| | MPZ, | | nerves/axons | |
| | MFN2 | | | |
| Tay-Sachs Disease | HEXA | Gene addition | Brain | Flotte et al., 2022 |
| | | therapy | | |
| GM1 gangliosidosis | GLB1 | Gene addition | Brain | Hocquemiller et |
| | | therapy | | al. 2022 |
| Sandhoff Disease | HEXB | Gene addition | Brain/Spinal Cord | Lahey et al. 2020. |
| | | therapy | | |
| Chronic | CRMP2 | Gene addition | Dorsal root | Gomez et al. |
| pain/neuropathic | | therapy | ganglion neurons | 2023. |
| pain | | | | |
| Aromatic L-amino | AADC | Gene addition | brain | Pearson et al. |
| acid decarboxylase | | therapy | | 2021 |
| (AADC) deficiency | | | | |

### Pharmaceutical Compositions and Methods of Administration

The methods described herein include the use of pharmaceutical compositions comprising the AAVs as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intraarterial, subcutaneous, intraperitoneal, intrathecal, intramuscular, or injection or infusion administration. Delivery can thus be systemic or localized. For example, for delivery into the inner ear, delivery into the cochlea through application over or through the round window membrane, through a surgically drilled cochleostomy adjacent to the round window, a fenestra in the bony oval window, or a semicircular canal can be used (see, e.g., Kim et al., Mol Ther Methods Clin Dev. 2019 Jan. 11; 13:197-204; Ren et al., Front Cell Neurosci. 2019; 13: 323); for delivery into the retina, subretinal or intravitreal injections can be used (see, e.g., Ochakovski et al., Front Neurosci. 2017; 11: 174; Xue et al., Eye (Lond). 2017 September; 31(9):1308-1316).

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration. For example, the kit can include compositions comprising an AAV comprising a peptide as described herein.

### Examples

The invention is further described in the following examples, which do not limit the scope of the invention described in the claims.

### Methods

The following materials and methods were used in the Examples below.

### Animal Experiments

Animal studies were performed at Biomere-Biomedical Research Models (Worcester, MA) according to animal use guidelines and approved procedures. The Test Facility is accredited by the Association for the Assessment and Accreditation of Laboratory Animal Care, International (AAALAC) and registered with the United States Department of Agriculture (USDA) to conduct research in laboratory animals.

Non-human primates: A total of four cynomolgus monkeys (Macaca fascicularis) were used in this entire study (see Table I for complete animal information).

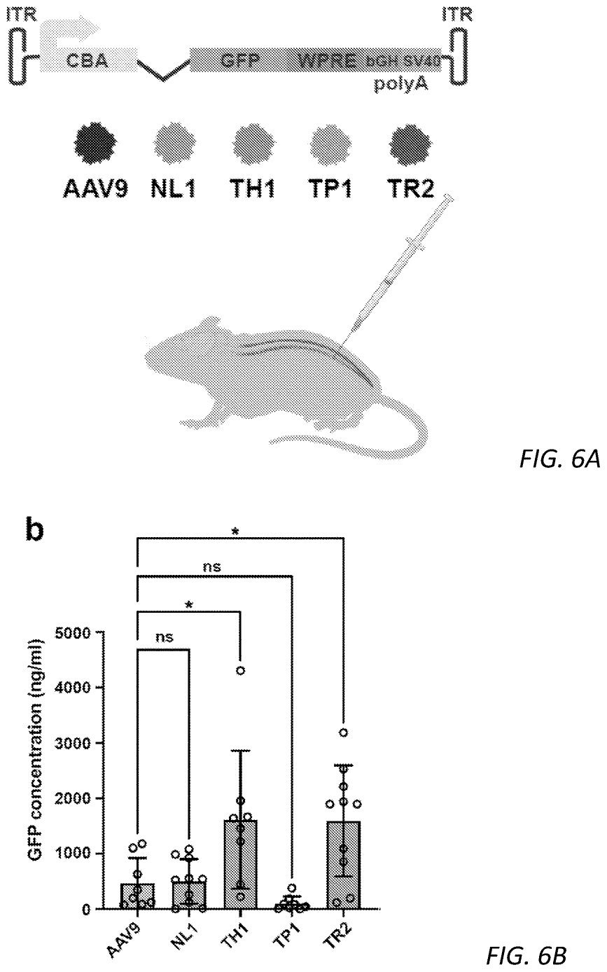

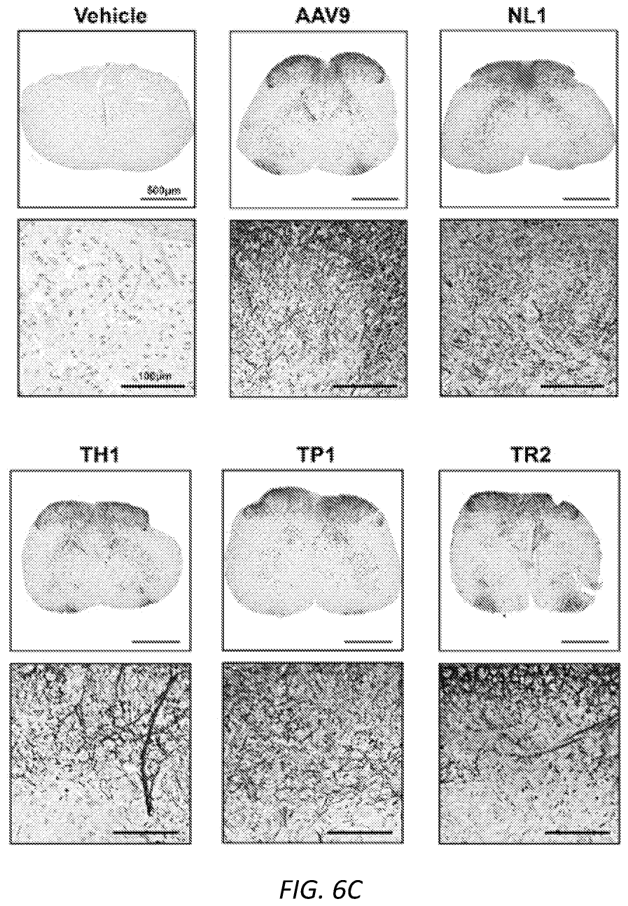

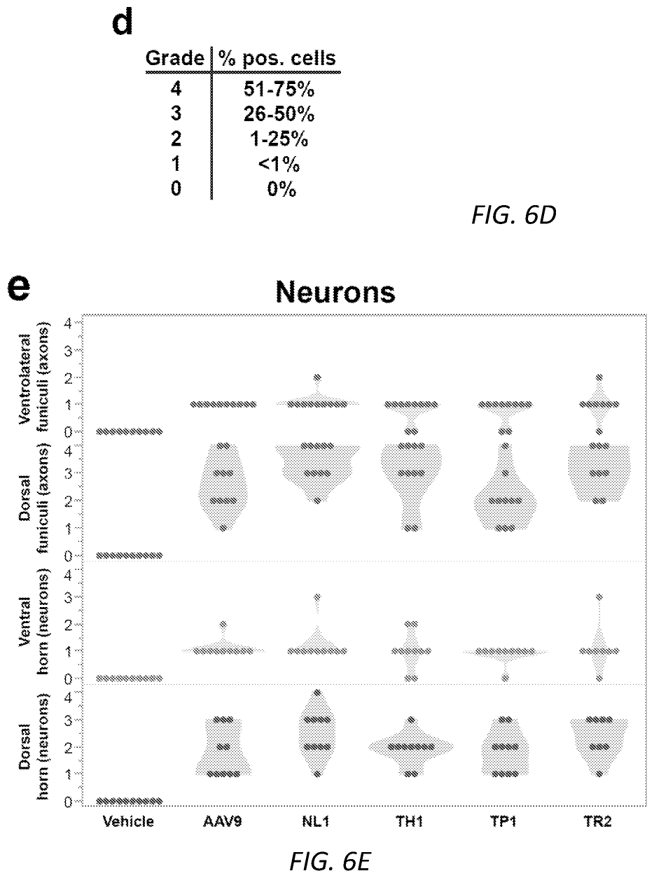

Mouse experiments: A total of 57 adult male C57BL/6 mice (Jackson Laboratories) were utilized for the data described in FIGS. 6A-D.

### Cell Culture.

Human 293T cells were obtained from American Type Culture Collection (Manassas, VA). Cells were cultured in high glucose Dulbecco's modified Eagle's medium containing HEPES (Invitrogen, Carlsbad, CA) supplemented with 10% fetal bovine serum (FBS) (Sigma, St. Louis, MO) and 100 U/mL penicillin, 100 g/mL streptomycin (Invitrogen) in a humidified atmosphere supplemented with 5% CO2 at 37° C.

### AAV Plasmid Constructs

The iTransduce plasmid, pAAV-CBA-Cre-mut/p41-Cap9 (containing the insert with 7mer peptide sequences has been previously described¹⁶.

pAAV-CBA-Floxed-STOP-H2B-mPlum plasmid was constructed by digesting the AAV ITR containing plasmid, AAV-CBA-WPRE, after the CBA promoter with HindIII and NheI restriction sites. A DNA fragment for a floxed transcription stop transcription site (3 copies of SV40 late poly A sequence) followed by a H2B protein fused to mPlum was synthesized by Genscript (Piscataway, NJ) and inserted into pUC57-Kanamycin plasmid. Next pUC57-Kan with Floxed-STOP-H2B-mPlum was digested with HindIII and NheI and inserted into the above digested AAV plasmid to create pAAV-CBA-Floxed-STOP-H2B-mPlum (plasmid electronic map available upon request).

pAAV-CBA-hFrataxin-HA-BC-WPRE plasmid was constructed by digesting the AAV transgene expression plasmid, pAAV-CBA-GFP, with AgeI and NheI to remove the GFP cDNA fragment. A double stranded DNA gBlock™ fragment was ordered from Integrated DNA Technologies (IDT, Coralville, Iowa) which contained human frataxin cDNA fused with a hemagglutinin (HA) tag on its C-terminus. This fragment was inserted into the pAAV plasmid above using the Gibson Assembly® Master Mix (New England Biolabs, Ipswich, MA). This “base” plasmid, pAAV-CBA-hFrataxin-HA was used as the acceptor plasmid to insert barcode fragments downstream of the HA sequence (plasmid map available on request). To insert the unique DNA/RNA barcode sequences into the base plasmid, pAAV-CBA-hFrataxin-HA was digested with XhoI. Next, we ordered double stranded DNA gBlock™ fragments (130 bp) from IDT which had unique 21mer bp barcodes (for 14 capsid candidates as well as AAV9) with flanking homology arms. These 15 different barcodes were individually cloned into the XhoI-digested pAAV-CBA-hFrataxin-HA plasmid using Gibson Assembly as before. Each barcoded pAAV-CBA-hFrataxin-HA plasmid was complete-plasmid sequenced at PlasmidSaurus (Eugene, OR).

AAV 9 capsid candidates containing unique 7mer peptides were individually cloned into the AAV9 rep/cap plasmid, pAR9, as previously described¹⁶. The plasmid AAV-CBA-GFP has been previously described³⁸ and was used in the individual capsid comparisons in mice and in human spinal cord organoids. This AAV expression plasmid encodes a single stranded AAV genome with inverted terminal repeat (ITR)-flanked transgene cassette with a CMV I/E enhancer, chicken beta actin promoter, a chimeric intron, a woodchuck hepatitis virus posttranscriptional regulatory element (WPRE), and tandem bovine growth hormone (BGH) and SV40 polyA signal sequences.

### AAV Library Production

Production of the AAV9 iTransduce library was performed as previously described¹⁶ with some changes. Fifty tissue culture dishes (15 cm diameter) were used (1.5×10⁷ 293T cells seeded per plate), with cells cultured in DMEM containing 10% fetal bovine serum (FBS) and 100 U/mL of penicillin, 100 μg/mL streptomycin, and 292 μg/mL L-glutamine (Invitrogen). Twenty-four hours after plating, cells were triple transfected using polyethylenimine (PEI) transfection reagent, adding to each 15 cm plate: pAAV-CBA-Cre-mut/p41-Cap9 (containing the insert with 7mer peptide sequences; 200 ng), pAR9-Cap9-stop/AAP/Rep (provides rep in trans, 7 μg), and pAdΔF6 (helper plasmid, 15 μg), and sheared salmon sperm DNA (6 μg). Seventy-two hours post transfection, AAV was isolated from a pool of clarified cell lysate and polyethylene glycol (PEG)-precipitated vector from the conditioned media. The pooled virus was then purified by iodixanol density-gradient ultracentrifugation. Buffer exchange to phosphate buffered saline (PBS) containing 0.001% Pluronic F-68 (Gibco) was done using ZEBA spin columns (7K MWCO; Thermo Fisher Scientific) and further concentration was performed using Amicon Ultra 100 kDa MWCO ultrafiltration centrifugal devices (Millipore). We quantified AAV vector genomes (vg) in AAV preparations using TaqMan qPCR with BGH polyA-sequence specific primers and probe²⁸. Endotoxin for both vector preparations was determined to be <1 EU/mL using Endosafe® LAL Cartridges and the Endosafe® nexgen PTS™ device (Charles River, Charleston, SC). Vector purity was assessed by silver staining of SDS PAGE gels in which 1×10¹⁰ vg of each vector was run/lane. Purity of both preparations were >90%. Vector was stored at −80° C. until use.

AAV-CBA-Floxed-STOP-H2B-mPlum production. This vector was produced, purified, and titered by Vector Biolabs (Malvern, PA). Endotoxin level was below 1 EU/mL.

### Barcoded AAV Capsid Candidate Production

We individually packed the AAV-CBA-hFrataxin barcoded genome in each capsid candidate and the AAV9 capsid. For each vector we transfected five tissue culture dishes (15 cm diameter, 1.5×10⁷ 293T cells seeded per plate), with cells cultured in DMEM containing 10% fetal bovine serum (FBS) and 100 U/ml of penicillin, 100 μg/mL streptomycin, and 292 μg/mL L-glutamine (Invitrogen). Twenty-four hours after plating, cells were triple transfected using polyethylenimine (PEI) transfection reagent, adding to each 15 cm plate: pAAV-CBA-hFrataxin-HA-barcode (6 μg), pAR9-peptide of interest (7 μg), and pAdΔF6 (helper plasmid, 15 μg). Seventy-two hours post transfection, AAV was isolated from a pool of clarified cell lysate and polyethylene glycol (PEG)-precipitated vector from the conditioned media. The pooled virus was then purified by iodixanol density-gradient ultracentrifugation. Buffer exchange to phosphate buffered saline (PBS) containing 0.001% Pluronic F68 (Gibco) was done using ZEBA spin columns (7K MWCO; Thermo Fisher Scientific) and further concentration was performed using Amicon Ultra 100 kDa MWCO ultrafiltration centrifugal devices (Millipore). We quantified AAV vector genomes (vg) in AAV preparations using TaqMan qPCR with BGH polyA-sequence specific primers and probe²⁸. We next pooled the barcoded vectors and endotoxin was determined to be <1 EU/mL using Endosafe® LAL Cartridges and the Endosafe® nexgen PTS™ device (Charles River). Pooled vector purity was assessed by staining SDS PAGE gels with GelCode™ Blue (ThermoFisher) in which 1×10¹¹ vgs were run/lane. The purity of both preparations were >90%. Vector was stored at −80° C. until use.

### AAV Capsid Production for Mouse and Human Spinal Cord Organoid Transduction.

AAV production was identical to that described in “barcoded AAV capsid candidate production” above with the exception that the expression plasmid was AAV-CBA-GFP.

### Non-Human Primate Library Selection.

Animals were considered acclimated to the environment at the time of transfer to the study. Prior to the study, serum from each animal was tested for neutralizing antibodies to AAV9 by the University of Pennsylvania Gene Therapy Program Immunology Core run by Dr. Jessica Chichester. Titers for all animals were at or below 1:5.

Round 1 selection. On Day 0, the animal received an IT dose of unselected AAV library as appropriate to group (per the study design Table I). The dose volume injected was 0.74 mL and vector dose of 9×10¹¹ vg. The animal was given Buprenorphine (0.03 mg/kg, IM) and Meloxicam (0.2 mg/kg, SC) prior to the procedure for the purpose of analgesia. The animal was sedated with Ketamine 7.5-12 mg/kg and Dexdomitor 0.01-0.03 mg/kg mixture, IM; Atipamezole (0.1-0.3 mg/kg, IM) was used for reversal. The animal was positioned in lateral recumbency while on a circulating warm water blanket and/or forced warm air blanket during the procedure. The head was kept in line with the spine and the hips and shoulders were perpendicular to the table. The lower back was arched to increase spacing between the spinous processes. The lumbosacral region (the area over ~L4/5 for cynomolgus) was clipped and aseptically prepared utilizing 3 alternating scrubs of either povidone iodine or chlorhexidine scrub solution and sponges soaked in 70% isopropyl alcohol. A line block (i.e., Lidocaine/Bupivacaine) ~0.20-0.50 mL, SC was administered at the lumbar puncture site. A final application of ChloraPrep™ or appropriate antimicrobial was applied to the puncture site and allowed to dry.

The wings of the ileum were palpated to provide anatomical landmarks. The two spinous processes were identified, and in between which the spinal needle (22 g×1.5″) was introduced. The skin was penetrated and needle slowly advanced. After confirmation of placement in the intrathecal space, ~0.5 mL of CSF was removed prior to dose administration. Next, the AAV vector was slowly administered over 1-2 minutes. After administering the dose, the syringe and needle was left in place for ~5 seconds and after removal, pressure applied to the injection site. Parameters were observed constantly throughout the procedure including heart rate, respiratory rate and oxygen saturation. Animals were recovered from anesthesia and moved to a recovery area, placed on a circulating warm water blanket and/or forced warm air blanket and covered with a dry towel. Animals were observed following the procedure and kept in the recovery area until the animal was conscious and able to hold itself in a sitting position. Animals were then transported to their home cage. Approximately 2 hours post vector dosing, animals were immunosuppressed with intramuscular dosing of 0.5 mg/kg of dexamethasone, which continued daily until necropsy at day 21.

Round 2 selection. The IT injection and animal care was identical to round 1 with the following changes. First the animal was lumbar-injected with the recovered and packaged round 1 library at a dose of 2×10¹¹ vg in a volume of 0.6 mL. CSF was collected before injection. Three hours later, the animal was IT-injected in a different lumbar region than the first dose with 3×10¹³ vg of AAV9-CBA-FLOXED-STOP-H2B-mPlum vector in a total volume of 0.6 mL.

### NHP Necropsy and Tissue Processing.

On Day 21±1 day, animals were anesthetized with Ketamine 7.5-12 mg/kg and Dexdomitor 0.01-0.03 mg/kg mixture. Nembutal was administered at 15-30 mg/kg. Once deeply anesthetized, the animal was perfused via left cardiac ventricle with cold heparinized (100 U/mL) saline until the outflow ran clear. Approximately 1 L of heparinized saline was used with a perfusion pump set to ~400 rpm. Euthanasia was performed per AMVA guidelines. For rounds 1 and 2, flash frozen samples were collected for the entire spinal cord and brain and stored at −80° C.

DNA Isolation from NHP Tissue During Selection Rounds

Whole tissue isolation. Spinal cord samples (0.5 cm-1 cm in length) were homogenized using 1.4 mm ceramic beads in a BeadBug tissue homogenizer (Benchmark Scientific, Sayreville, NJ) in buffer ATL of the DNeasy Blood and Tissue Kit (Qiagen). After homogenization, we followed the manufacturer's instructions to purify DNA. DNA concentration was determined using a NanoDrop spectrophotometer (ThermoFisher).

Nuclei isolation and flow sorting. Spinal cord samples were dissociated using a GentleMACS Dissociator (Miltenyi Biotec, Westphalia, Germany) using Nuclei Extraction Buffer (Miltenyi) and homogenate filtered through a 30 μm Smart Strainer (Miltenyi). Nuclei were next purified using a 30% OptiPrep™ iodixanol (Axis-Shield, Oslo, Norway) gradient (iodixanol serves to purify away cellular debris and the nuclei are pelleted to the bottom of the tube). The homogenate containing nuclei was layered on top of the iodixanol gradient and centrifuged at 10,000×g at 4° C. for 30 min in a fixed angle rotor (FA-45-6-30, Eppendorf, Enfield, CT). The gradient was carefully aspirated leaving the nuclei pellet in the tube. Nuclei were resuspended in 500 μl of cold PBS. Next nuclei were labeled with Vybrant™ DyeCycle™ Violet Stain (ThermoFisher) and were sorted on a BD FACS Aria II Cell Sorter (Becton Dickinson, Franklin Lake, NJ). First, violet stain positive nuclei were gated on (PI) using Pacific Blue-A laser. Then singlets were selected using FSC-A and FSC-H axes (P2). Finally, we selected mPlum positive nuclei using a PE-Cy5-A laser. Nuclei (mPlum positive or negative) were sorted into tubes and stored at −80° C. until DNA isolation. For DNA isolation, we used the Arcturus® PicoPure® DNA Extraction Kit (ThermoFisher).

### Next Generation Sequencing of Library.

Next generation sequencing was performed on the plasmid AAV9 library pool, as well as following packaging of capsids. Before injection, packaged naïve library was amplified by PCR and sequenced at a depth of ~105 reads to ensure adequate read depth and a lack of bias. Minimum requirements were >95% unique variant reads, and no single variant appearing in more than 10 sequencing. Sequencing was also performed following PCR rescue of the cap gene fragment (either from NHP spinal cord tissue or from nuclei sorted by flow cytometry). For each round of selection vector DNA corresponding to the insert-containing region was amplified by PCR using either Phusion High-Fidelity enzyme or Q5 polymerase (both from New England Biolabs using Forward primer: 5′-AATCCTGGACCTGCTATGGC-3′ (SEQ ID NO:2), and reverse primer: 5′-TGCCAAACCATACCCGGAAG-3′ (SEQ ID NO:3)). PCR products were purified using a QIAquick PCR Purification Kit (Qiagen). Unique barcode adapters were annealed to each sample, and samples were sequenced on an Illumina MiSeq (150 bp reads) at the Massachusetts General Hospital Center for Computational and Integrative Biology DNA Core. Approximately 50,000-100,000 reads per sample were analyzed. Sequence output files were quality-checked initially using FastQC (bioinformatics.babraham.ac.uk/projects/fastqc/) and analyzed on a program custom-written in Python. Briefly, sequences were binned based on the presence or absence of insert; insert-containing sequences were then compared to a baseline reference sequence and error-free reads were tabulated based on incidences of each detected unique insert. Inserts were translated and normalized.

Non-human primate candidate barcoded library screen. Two adult male cynomolgus monkeys (see Table I for NHP information) were intrathecally injected in the same manner as for the selection, except with 4.48×10¹² vg of the pooled barcoded capsids. Three weeks later, animals were perfused with sterile heparinized saline. Samples for paraffin embedding were fixed in 10% neutral buffered formalin before transferring the samples to PBS. Samples for cryosectioning were fixed in 4% buffered formaldehyde for 24 h before transferring samples to PBS. Samples for DNA and RNA extraction were immediately frozen on dry ice and stored at −80° C.

DNA and RNA Isolation from NHP Whole Tissue for Barcoded AAV Capsid Candidates

DNA isolation. Whole tissue isolation. Spinal cord (and other tissue) samples (0.5 cm-1 cm in length) were homogenized using 1.4 mm ceramic beads in a BeadBug tissue homogenizer (Benchmark Scientific) in buffer ATL of the DNeasy Blood and Tissue Kit (Qiagen). After homogenization, we followed the manufacturer's instructions to purify DNA. DNA concentration was determined using a NanoDrop spectrophotometer (ThermoFisher Scientific).

RNA isolation. Spinal cord (and other tissue) was placed into Qiazol (Qiagen) and subjected to homogenization with 1.4 mm ceramic beads in a BeadBug tissue homogenizer (Benchmark Scientific). After performing a phenol/chloroform extraction of RNA, the RNA was precipitated using isopropranol and centrifugation, followed by a 75% ethanol wash. The RNA pellet was resuspended in RNase-free water and stored at −80 C until further processing. The RNA sample was further purified using RNeasy spin columns (Qiagen) and the sample was eluted in RNase-free water. Next residual DNA contamination was removed using DNaseI treatment with a DNA-free™ kit (Ambion by Life Technologies). cDNA was synthesized from RNA (approximate input 500 ng of RNA) using the SuperScript™ IV VILO™ Master Mix reverse transcriptase (RT) kit. For each sample we did a control where the RT enzyme was left out to ensure the PCR amplicons originated from RNA and not contaminated DNA in the RNA samples.

Quantitative PCR to measure absolute amounts of AAV genomes and transgene mRNA in spinal cord of pooled AAV injection in NHPs. DNA and RNA isolation and cDNA synthesis from NHP spinal cord was performed as described immediately above. To determine the amounts of AAV vector genomes or transgene expression (frataxin-HA) we used the same Taqman probes and primers targeting BGH polyA used to titer our AAV vectors. For AAV vector genomes, we used a standard curve with an AAV plasmid to perform absolute quantitation. We used 54 ng of genomic DNA template from lumbar, thoracic, and cervical spinal cord as input for the qPCR. A separate qPCR reaction for each sample was performed to detect the NHP gene UBE2D2 (ubiquitin conjugating enzyme E2 D2) using the Taqman Gene Expression Assays 20× mix (Catalog #4351372, assay ID Mf07285893_s1, ThermoFisher Scientific). AAV genomes were inferred from the AAV plasmid standard curve and adjusted to AAV genomes/μg of genomic DNA. For each AAV genome sample we calculated the 2\hat{ }^{delta Ct}(sample Ct−sample with lowest Ct) of the UBE2D2 qPCR sample and then normalized the AAV genomes to that value. This compensated for DNA input differences for each sample.

For the RT-qPCR samples, we used the AAV plasmid as a standard curve as above to calculate absolute amounts of cDNA. We used 1 μl of the RT reaction from mRNA isolated from NHP lumbar spinal cord as template for the qPCR. A separate qPCR reaction for each sample was performed to detect the NHP cDNA of GAPDH (glyceraldehyde-3-phosphate dehydrogenase) using the Taqman Gene Expression Assays 20× mix (assay ID Mf04392546_g1, ThermoFisher Scientific). This probe spans exons, so it is selective for cDNA detection over genomic DNA. cDNA values for AAV transgene were normalized to the respective GAPDH Ct values for each sample to control for differences in input total cDNA. All qPCR reactions were performed in a 7500 FAST qPCR system (Applied Biosystems).

Quantitative PCR to measure absolute amounts of AAV genomes in spinal cord organoids. We used the same protocol as above with for “Quantitative PCR to measure absolute amounts of AAV genomes and transgene mRNA in spinal cord of pooled AAV injection in NHPs” with the following modifications. DNA was extracted and purified from organoids using the DNeasy Blood and Tissue Kit (Qiagen). The same Taqman probe and primer set described above was used for AAV genomes. For human cDNA detection used for normalization we used a probe and primer set specific for the human ubiquitin conjugating enzyme E2 D3 (UBE2D3) Taqman Gene Expression Assays 20× mix (Assay Id Hs00704312_s1, ThermoFisher Scientific).

PCR to amplify barcodes for NGS. For both the DNA and RNA samples from the pooled barcoded library injected animals we used the following reagents. A PCR using either the DNeasy-purified DNA or the cDNA from the RT reaction as template was performed (usually 150 ng DNA template input). We used Q5 polymerase (NEB) and primers that amplify a 182 bp amplicon which contains the unique barcode for each capsid. Primers were: SC-Lib-Fwd (5′ gatgcttacccttacgacgt 3′) and SC-Lib-Rev (5′ cagcgtatccacatagcgta 3′). The PCR product was purified using the PureLink™ PCR Purification Kit (ThermoFisher Scientific) and samples were submitted for NGS as described above. We initially sequenced an aliquot of the pooled barcoded library to ascertain the precise frequencies of the barcoded variants on input. Output frequencies in all tissues were normalized to these initial ratios. Insert-containing sequence reads were binned using Hamming distance, comparing to the known barcode sequences; additional quality control was undertaken to ensure only true barcoded reads were assigned to each capsid.

Intrathecal lumbar injection in mice. Adult (6-8 weeks old) male C57BL/6 mice received a single intrathecal dose of each of the four vector groups (n=5/group) described in FIG. 6 or control treatment (vector formulation buffer). Experimenters were blinded to the vector identity. Animals received a prophylactic dose of Buprenorphine SR (1 mg/kg, SC), then were anesthetized by isoflurane to effect. Syringes were loaded individually prior to each dose with 10 L of dosing solution per animal. The test material was delivered manually, using an insulin syringe with a 28-30-gauge needle, by intrathecal injection to the lumbar spine at the L4-L5 or L5-L6 intravertebral space. The tip of the needle was introduced into the lumbar spine, and proper insertion of the needle was confirmed by tail flick reflex. After slowly injecting the total dose volume, the needle will be allowed to stay in place for a minimum of 5 seconds to avoid efflux of the test material to periphery. Animals were placed on a circulating water heating pad until fully recovered from anesthesia. Four weeks after administration, animals were euthanized (carbon dioxide asphyxiation, to effect, in conjunction with exsanguination), and tissues were removed for fixation (histology) or snap frozen in liquid nitrogen.

Immunohistochemical staining of mouse tissues from mice injected with AAV capsids packaging AAV-CAG-GFP cassette. Tissues designated for histologic processing were placed in appropriately sized histology cassettes and fixed in 10% NBF (neutral buffered formalin) at ambient temperature for 24-48 hours then transferred to 1×PBS and stored at 5° C.±3° C. until embedded.

The immunohistochemical staining of GFP in spinal cords and livers of the injected mice was performed by the Penn Vet Comparative Pathology Core (University of Pennsylvania, Philadelphia, PA). Briefly, the formalized fixed tissue as paraffin embedded and 5 μm thick paraffin sections mounted on ProbeOn™ slides (Thermo Fisher Scientific). The immunostaining procedure was performed using a Leica BOND RX^{m} automated platform combined with the Bond Polymer Refine Detection kit (Leica DS9800). Sections on slides were dewaxed and rehydrated in deionized water and then pretreated with the epitope retrieval BOND ER2 high pH buffer (EDTA Based pH=9.0, Leica AR9640). Next endogenous peroxidase was inactivated with 3% H2O2. Nonspecific protein-protein interactions were blocked with Leica PowerVision IHC/ISH Super Blocking solution (Leica PV6122) at RT. The primary rabbit polyclonal anti-GFP antibody (Cat. no. A11122, ThermoFisher) was diluted 1/1000 using Cell Signaling Technology (CST) diluent solution and was applied for 45 minutes at RT. A biotin-free polymeric IHC detection system (Leica DS9800) consisting of HRP conjugated Gt anti-Rb IgG secondary antibody was applied for 25 minutes at RT. Immunoreactivity was detected with the diaminobenzidine (DAB) chromogen reaction. Slides were counterstained in hematoxylin, dehydrated in ethanol series, cleared in xylene, and mounted with resinous mounting medium (Thermo Scientific, ClearVue™ coverslipper). Examination and grading of the GFP staining in cell types (e.g., neurons, glia) was performed by trained personnel at the core facility.

GFP enzyme-linked immunosorbent assay (ELISA) of spinal cord from mice injected intrathecally with AAV capsids packaging AAV-CAG-GFP cassette. A GFP ELISA kit (abcam ab171581) was used to detect AAV vector encoded GFP in spinal cord homogenates. Flash-frozen spinal cord segments (each spinal cord was divided into a caudal and rostral segment/mouse) were cut into pieces weighing approximately 5-20 mg and placed into individual tubes with a single stainless-steel bead. Next, we added cell extraction buffer from the kit to each tube and homogenized the tissue in a BeadBug™ benchtop tissue homogenizer (Benchmark Scientific, Atkison, NH) for 5 s. Next, protein concentrations were determined for each sample using a bicinchoninic acid (BCA) Protein Assay Kit (Cat. No. 23227, Pierce, Thermo Fisher Scientific). Samples were all adjusted to the same concentration by diluting to the sample with the lowest concentration. Next, we performed the ELISA according to the manufacturer's instructions at a dilution of sample that fell within the range of the standard curve of the assay (sensitivity 18.8-1,200 μg/ml). Assay operators were blinded to the identity of the samples until data was plotted and graphed.

### Spinal Cord Organoids

### Organoid Laboratory #1:

### Production of Spinal Cord Organoids and Transduction

Spinal cord organoids were generated as previously described³¹. hESCs (H9) were grown in the mTeSR1 (STEMCELL Technologies) medium in 35 mm-diameter tissue culture dishes coated with Matrigel (Corning, 354277; 1:25 in DMEM/F12). hESCs were detached from 35 mm-diameter dishes using ReLeSR (STEMCELL Technologies), and cell aggregates in mTeSR were added to each well of a 12-well micropattern plate. The following day, mTeSR medium was replaced with the neural cell induction medium (DM, DMEM/F12, 1% N2 supplement, 2% B27 supplement, 1% NEAA, 1% penicillin, and 0.1% 2-mercaptoethanol) containing 10 μM SB431542 (R&D) and 3 μM CHIR99021 (Sigma), and was subsequently replaced every day. After 3 days, colonies were detached using the pressure of a cell recovery solution (Corning, Cat #0.354253) from the pipette under a stereoscopic microscope and transferred into an Ultra-Low Attachment 96-well clear round bottom plate (Corning-Costar). Further, they were further grown in 3D with DM containing 20 ng/mL bFGF. After 6 days of bFGF treatment, 3D structures were grown without bFGF in the neural cell induction medium, and further grown in mixture of neurobasal medium and DM. After 30 days, organoids were transferred to 6 well plate and grown while shaking 80 rpm. For AAV transduction, organoids of 66 days or 225 days were transferred into an Ultra-Low Attachment 96-well plate. 35 μl of 1.17×10¹¹ gc of AAV in PBS containing 10% sucrose was added to each organoid³³. After 1-hr incubation at 37° C. incubator, the organoids were transferred to 6 well plate and incubated while shaking 80 rpm. After six days, live images of each organoid were taken in 5-μm steps along the z-axis using confocal microscope (Leica TCS SP8 confocal microscope). Stacked images using Z stack maximal projection were used for quantification using ImageJ.

### Immunostaining

Spinal cord organoids were fixed using 4% paraformaldehyde in 0.1 M phosphate-buffered saline (PBS; pH 7.4) for 1 hr at 25° C. Organoids were washed three times with PBS and incubated with 30% sucrose in PBS at 4° C. overnight, embedded in Tissue-Tek Optimal Cutting Temperature (OCT. Compound, SAKURA), frozen on dry ice, and cryosectioned. Sliced samples on New Silane IIWE coating slide (Muto Pure Chemicals) were blocked with 0.2% Triton X-100 with 3% bovine serum albumin (BSA) in PBS for 30 min at 25° C. Subsequently, samples were incubated with primary antibodies in a blocking buffer overnight at 4° C. Samples were washed three times with PBST (0.2% Triton X-100 in PBS) and incubated with secondary antibodies, with Hoechst for cell nuclei staining (1:2,000), in the blocking buffer for 1 h at 25° C. After washing with PBST, samples were mounted in Crystal Mount (Biomed) and imaged using a confocal microscope (Leica TCS SP8 confocal microscope).

### Antibodies:

GFP (1:2000, Abcam, ab13970)
NeuN (1:1000, Synaptic systems, 266 004)
Internexin (1:1000, Novus, NB200-140)
SOX2 (1:500, Milipore, AB5603)
GFAP (1:1000, DAKO, z0334)

### Fluorescence Intensity Measurement

To quantify the fluorescence throughout the organoid samples the original TIFF files were loaded into ImageJ and the channels were split into two windows. For each window (DAPI and GFP) the Slice Keeper (Image>Stacks>Tools>Slice Keeper) function was used to extract every 7^{th} slice from the stacks. The brightness and contrast for the DAPI stack was adjusted to be able to visualize the whole perimeter of the organoid for each slice while all the slices in the GFP stack were set to the same minimum and maximum displayed value (Min: 12 and Max: 54). To use the DAPI channel as the region of interest the windows needed to be synchronized (Analyze>Tools>Synchronize Windows). After synchronizing the windows, the freehand selection cursor was used to outline the signal of the DAPI channel. The following measurements were acquired: Area, Mean Gray Value, Min & Max Gray Value and Integrated Density. All data was saved in Excel spreadsheets.

Cell counting. We counted GFP positive cells in the confocal z-stacks images for 3 organoids/capsid. We used the Cell Counter Plugin. For each sample we counted GFP+ cells in the 7^{th}, 25^{th}, and 49^{th} slice of the z-stack. All GFP positive cells were manually selected in each slice and counted with the software and exported into Excel spreadsheets.

### Spinal Cord Organoid Laboratory #2

### Spinal Cord Organoid Generation

Spinal cord organoids were generated from hiPSCs as previously described³⁹ with the following modifications. In short, hiPSC colonies cultured on vitronectin (Stem Cell Technologies, #7180) and in TeSR™-E8™ medium (Stem Cell Technologies, #5940) were dissociated with Accutase (MERCK, #SF006) at 37° C. for 7 min., made into a single-cell suspension, and 9,000 cells were plated into individual ultra-low attachment U-bottom 96-well plates (Corning, #CLS7007) in 150p of TeSR™-E8™ medium (Stem Cell Technologies, #5940) supplemented with ROCK inhibitor (10 μM; Selleckchem, #S1049) and FGF2 (4 ng/ml; Peprotech, #AF-100-18B). On day 2, 150 μl of TeSR™-E8™ medium supplemented with the two SMAD inhibitors dorsomorphin (2.5 μM; Selleckchem, #S7306) and SB431542 (10 μM; Selleckchem, #S1067) was added. Two thirds of this medium was refreshed daily for the next five days, supplementing with CHIR 99021 (3 μM; Cayman Chemicals, #13122) from day 6 to 20. On day 7, medium was changed to Neural Maintenance Medium (NMM) consisting of 1:1 DMEM/F12 (Thermo Fisher Scientific, #11330) and Neurobasal Medium (Thermo Fisher Scientific, #10888) supplemented with 0.5% N2 (Thermo Fisher Scientific, #175020), 1% B27 (Thermo Fisher Scientific, #17504), human insulin (5 μg/ml; MERCK, #I9278), L-glutamine (1.5 mM; Thermo Fisher Scientific, #25030), non-essential amino acids (100 μM; Thermo Fisher Scientific, #11140), β-mercaptoethanol (10 μM; Thermo Fisher Scientific, #21985023), and penicillin-streptomycin (100 U/ml; Thermo Fisher Scientific, #15140), and supplemented with EGF (20 ng/ml; Peprotech, #AF-10015), FGF-2 (10 ng/ml; Peprotech, #AF-100-18B), and RA (0.1 μM, ReproCell, #04-0021) until day 20, with the addition of SAG (0.1 μM; Cayman Chemicals, #11914) from day 13. From day 8 on, medium was refreshed every other day. On day 21, spinal cord organoids were moved to Poly(2-hydroxyethyl methacrylate)-treated (MERCK, #P3932) 24-well plates (VWR, #734-2325), one organoid per well, in NMM supplemented with NT3 (20 ng/ml; Peprotech, #450-03), BDNF (20 ng/ml; Peprotech, #450-02), IGF1 (10 ng/ml; Peprotech, #100-11), cAMP (1 μM; MERCK, #D0260), and Ascorbic acid (150 μM; MERCK, #A4544), with three quarters medium refreshments every three to four days. From day 35 onward, spinal cord organoids were maintained in NMM supplemented with PDGF (10 ng/ml; Bio-techne, #221-AA), IGF1 (10 ng/ml), T3 (60 ng/ml; MERCK, #T6397), cAMP (1 μM), and Ascorbic acid (150 μM).

### Spinal Cord Organoid Transduction

On day 45, two thirds of medium (total of 1 ml) was refreshed with the addition of 2.5×10¹⁰ vg/organoid of AAV9, NL1, TH1, TP1, and TR2 capsids. GFP signal was monitored in live-organoids and medium was refreshed every three to four days. 10 days after transduction, organoids were collected for DNA isolation and immunohistochemistry (IHC).

Generation of hiPSC-Derived Motor Neurons

hiPSC-motor neurons were generated following⁴⁰ with the following modifications. Briefly, hiPSC colonies cultured on vitronectin (Stem Cell Technologies, #7180) and in TeSR™-E8™ medium (Stem Cell Technologies, #5940) were dissociated with Accutase (MERCK, #SF006) at 37° C. for 5 min., made into a single-cell suspension, and 9,000 cells were plated into individual ultra-low attachment U-bottom 96-well plates (Corning, #CLS7007) in 150p of TeSR™-E8™ medium (Stem Cell Technologies, #5940) supplemented with ROCK inhibitor (10 μM; Selleckchem, #S1049) and FGF2 (4 ng/ml; Peprotech, #AF-100-18B). On day 2, 150 μl of NMM supplemented with CHIR 99021 (3 μM; Cayman Chemicals, #13122), SB-431542 SB431542 (2 μM; Selleckchem, #S1067), Dorsomorphin (0.2 μM; Selleckchem, #S7306), and Ascorbic acid (0.1 mM; MERCK, #A4544), was added (motor neuron progenitor media 1). On day 8, MNP1 was changed to MNP2 consisting of NMM supplemented with CHIR (1 μM), 2 μM SB-431542 (2 μM), Dorsomorphin (0.2 μM), RA (0.1 μM, ReproCell, #04-0021), SAG (0.25 μM; Cayman Chemicals, #11914), and Ascorbic acid (0.1 mM). Two thirds of medium was changed daily from day 2 to day 7, and every other day from day 8 onwards. On day 14, patterned EBs were dissociated into single cells with Accutase at 37° C. for 15 min. in rotation and plated in geltrex-coated plates at a cell density of 70,000 cells/cm² in MNP1 media (supplemented with 10 μM ROCK inhibitor on the plating day). On day 22, patterned progenitors were dissociated into single cells with Accutase at 37° C. for 5 min. and plated on Poly-L-Ornithine and laminin coated glass coverslips at a cell density of 35,000 cells/cm² in motor neuron differentiation (MN) media consisting of NMM supplemented with RA (0.5 μM), SAG (0.1 μM), Ascorbic acid (0.1 mM), cAMP (1 μM), BDNF (20 ng/ml), NT3 (10 ng/ml), GDNF (10 ng/ml), IGF1 (10 ng/ml), and DAPT (10 μM) (supplemented with 10 μM ROCK inhibitor on the plating day). From day 22 onwards, half of the media was changed every 3 to 4 days for the rest of the protocol.

### Motor Neuron Transduction

On day 28, 10⁵ vg/cell of AAV9, NL1, TH1, TP1, and TR2 capsids were added to the motor neuron cultures (12 well plates in 1 ml/well). GFP signal was monitored in live-neurons and medium was refreshed every three to four days. 6 days after transduction neurons were fixed in 4% PFA.

### Immunohistochemistry

Briefly, organoids were washed with PBS, fixed in 4% paraformaldehyde (PFA in dH₂O, ProSciTech, #C004) at room temperature for 30 min., and followed by sucrose cryopreservation (30% sucrose in PBS at 4° C. for 48 h), embedding in OCT (Tissue-Tec Oct Compound, #4583) and snap-freezing. For IHC, 20 μm thick sections were cut using a cryostat (Leica). After 6×PBS washes of 5 min., antigen retrieval was performed by incubating slides in 0.01M citrate buffer at 90° C. for 30 min. After one PBS wash, slides were blocked for 1 h at room temperature with blocking buffer consisting of PBS+goat serum (5%; Thermo Scientific, #16210)+BSA (0.1%; MERCK, #A9418)+Triton X-100 (0.3%, MERCK, #T8787). Primary antibodies in blocking buffer were incubated 1 h at room temperature and then overnight at 4° C. The next day, after 6×PBS washes of 5 min., organoids were incubated with secondary antibodies (1:1000; Thermo Scientific, Alexa Fluor-488, 594, 647) for 2 hours at room temperature. Afterwards, cells were washed 6×5 minutes with PBS, incubated with diamidino-2-phenylindole (1:1000; DAPI; MERCK, #D9542) for 2 minutes at room temperature, washed once with PBS and embedded with Fluoromount-G (Southern Biotech, #0100-01).

Motor neurons were washed with PBS, and gradient fixed in 2% PFA at room temperature for 20 min., followed by 4% incubation for another 20 min. For immunocytochemistry (ICC), cells were washed 6× with PBS, and blocked for 1 h at room temperature with blocking buffer. Primary antibodies in blocking buffer were incubated 1 h at room temperature and then overnight at 4° C. The next day, after 6×PBS washes of 5 min., cells were incubated with secondary antibodies (1:1000; Thermo Scientific, Alexa Fluor-488, 594, 647) for 2 h at room temperature, washed once with PBS and embedded with Fluoromount-G.

### Antibodies:

Anti-GFP (1:1000; Aves Labs, #GFP-1020)
ISL1 (1:2500; Abcam, #ab109517)
NeuN (1:500; Synaptic Systems, #266006)

### Organoid and Neuronal Imaging and Analysis

Confocal images of organoids were acquired with a Nikon ECLIPSE Ti inverted microscope (Nikon Corporation) controlled by NIS-Elements 4.30 software (Nikon Corporation) and fluorescent images of motor neurons were acquired with a Leica DMi8 inverted light microscope (Leica Microsystems) controlled by LAS X 3.7 software (Leica Microsystems). GFP+ cells in organoids and motor neurons were analysed using CLIJ2 plugin for Fiji³⁷. Briefly, within CLIJ2 GFP+ cells in both organoids and motor neurons were label by applying a subtracting a Gaussian Blur2D filter>Thresholding>Parametric Watershed>Connected components labeling Box>Duplicate and go ahead in Fiji. Next, GFP+ somas were quantified in Fiji. All analysis were performed blinded.

### Data Analysis

To assess common amino acids across the 7-mer insert between the 13-chosen candidates, we used the web-based application WebLogo (weblogo.threeplusone.com/)^{29, 30} which generates alignments of the input sequences, whereby the height of each amino acid corresponding to its frequency at that position.

### Statistics

We used GraphPad Prism 9.0 for PC for statistical analysis. For comparison of biodistribution (AAV genomes) between animals and across regions we used an ANOVA followed by a Šidik's multiple comparisons test. To compare cDNA levels of AAV transcripts between NHPs #1001 and #1002 in the lumbar region, we used an unpaired two tailed t-test; p values <0.05 were accepted as significant.

### Example 1. In Vivo Intrathecal Injection AAV Library Selection Strategy in NHPs

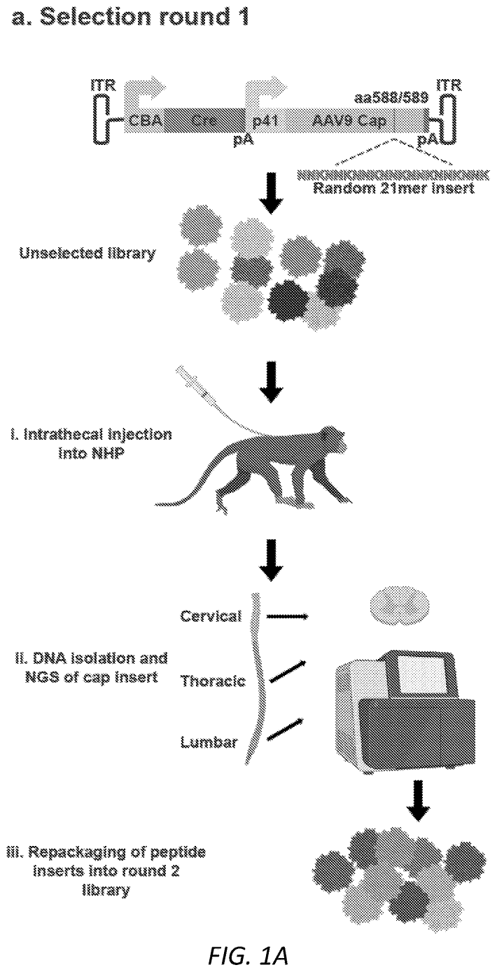

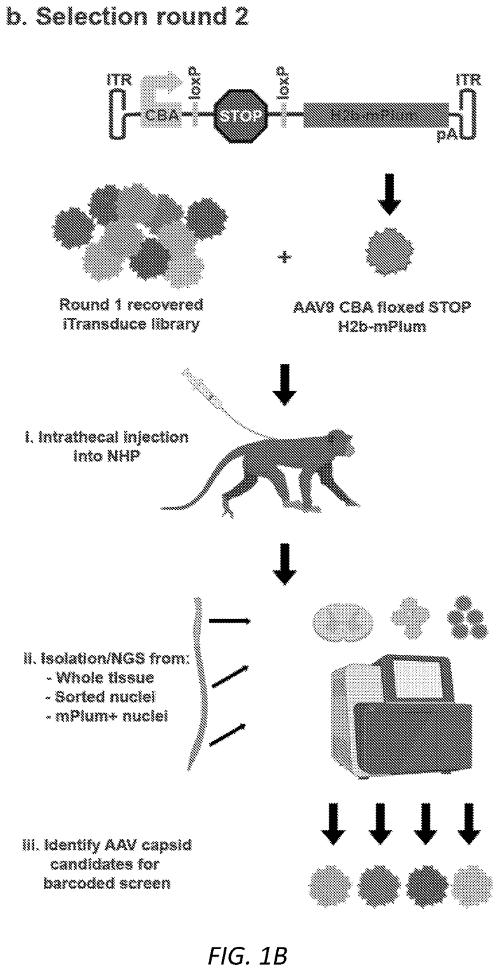

We have previously reported on the use of the iTransduce AAV peptide display library. The AAV genomes consists of a promoter driving a Cre-recombinase cassette as well as p41 promoter driven AAV9 capsid with 7-mer peptide inserts between amino acids 588-589 of VP1. This allows surface display of 50 copies of peptides on VP3 on the capsid surface (and 5 copies each on internally localized VP1 and VP2 proteins). For round one of our in vivo selection strategy in NHPs, we produced the AAV peptide display library and injected it intrathecally into Old World monkeys (cynomolgus macaques) (FIG. 1a, Table I). Next, AAV genome DNA was recovered by PCR from cervical, thoracic, and lumbar regions of the spinal cord and subjected to next generation sequencing (NGS) to analyze the content of the 7-mer encoding inserts. These capsid inserts were pooled and packaged into the round 1 selected spinal cord library (FIG. 1a). For the second selection round, we developed a strategy to enable selection of capsids that could mediate transduction expression in a non-transgenic animal such as an NHP. To do this we used a two-vector approach as outlined in FIG. 1b. The first vector is the AAV iTransduce library genome shown in FIG. 1a, that is packaged inside the round 1 rescued capsid inserts. The second vector is a Floxed reporter cassette encoding an H2B-fused mPlum protein called AAV-CBA-Floxed-STOP-H2B-mPlum. We used H2B-mPlum (mPlum with a nuclear localization signal) to allow sorting of fluorescent nuclei from either fresh or frozen tissue (cytoplasmic proteins are likely to leak out of freeze/thawed tissues). When the AAV capsid library encoding Cre is co-injected or sequentially injected with the AAV9-CBA-floxed-STOPH2B-mPlum, cells that are co-transduced with library capsids able to express Cre along with the AAV9 capsid will excise the stop cassette and express H2B-mPlum in the nucleus (FIG. 1b). Spinal cord is isolated, and DNA can be purified from whole frozen tissue, purified nuclei, or H2BmPlum flow-sorted nuclei. Peptide inserts are analyzed by NGS and candidate AAV capsids chosen for further characterization.

### Example 2. Round 1 of Selection of Intrathecally Injected AAV9 Peptide Display Library in NHPs

A preparation of the iTransduce AAV9 peptide display library was produced, purified, and titered. Library diversity was determined by NGS to be adequate for the selection and a male cynomolgus monkey was injected intrathecally with 9×10¹¹ vector genomes (vg) of the AAV library. Three weeks later, the animal was killed, perfused with sterile saline and tissues including the spinal cord were flash frozen and stored at −80° C. We isolated DNA from homogenized, whole, spinal cord samples from the cervical, thoracic, and lumbar regions. Next, we performed a PCR with primers surrounding the 21-mer inserts encoding the 7-mer peptides and submitted the three samples for low-depth (~10⁵ reads) NGS. We obtained diverse inserts from the sequencing data; reads and unique inserts frequencies are shown in Table II.

Read diversity (number of unique reads) in the cervical sample was much lower than thoracic or lumbar. This is likely due to few capsids with the ability to traffic from the lumbar region to the cervical region after intrathecal injection. We pooled amplified capsid DNA fragments containing 21-mer inserts from all three spinal cord regions and ligated them into the iTransduce plasmid library backbone.

### Example 3. Two Vector System Allows Detection of Transduction Competent AAV Capsids in Non-Transgenic Large Animals

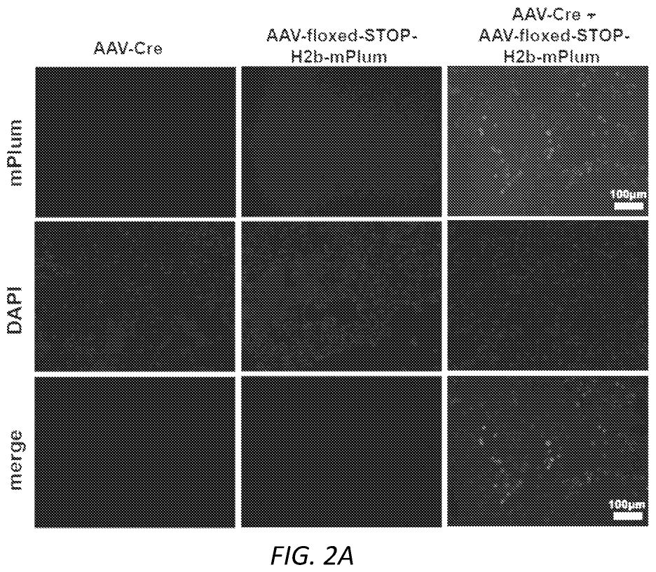

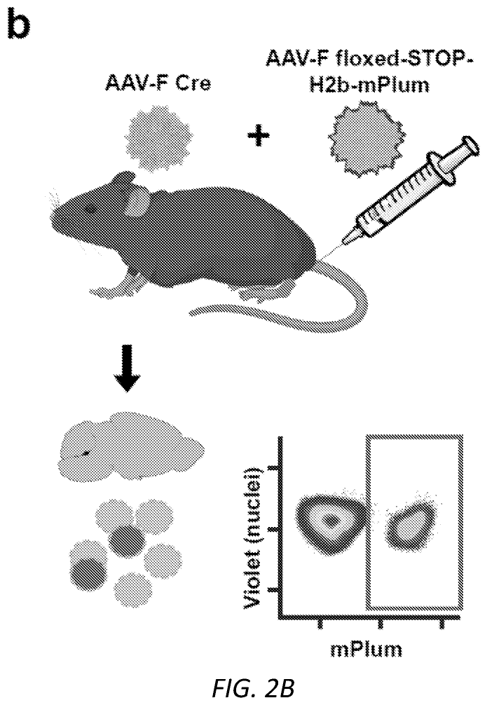

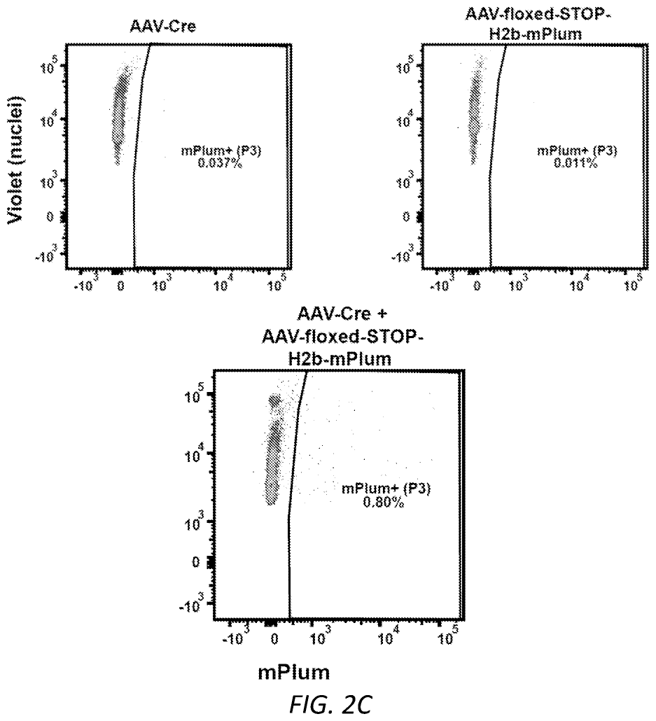

To create a system that would allow us to detect transgene expression at the protein level of transduction-competent AAV library variants, we designed a second vector, AAV-CBA-Floxed-STOP-H2B-mPlum as described in FIG. 1b. To test the function of the system we transduced cultured 293T cells with AAV capsids packaging the following expression cassettes: (1) AAV-PHP.B-CBA-Cre only, (2) AAV9-floxed-STOP-H2B-mPlum only, (3) mixture of AAV-PHP.B-CBA-Cre with AAV9-floxed-STOP-H2B-mPlum only. Three days later, cells were examined by fluorescence microscopy for both DAPI and H2B-mPlum fluorescence was detected only in the cells co-transduced with both vectors (FIG. 2a). To test whether the system worked in non-transgenic animals in vivo, we intravenously injected wild type C57BL/6 mice with the following groups of vectors: (1) AAV-F-CBA-Cre only, (2) AAV-F-Floxed-STOP-H2B-mPlum only, or (3) a mixture of AAV-F-CBA-Cre with AAV-F-Floxed-STOP-H2B-mPlum only. We used the previously described AAV-F capsid as it efficiently transduces the brain after systemic injection^{16, 17}. Sixteen days post injection, mice were perfused with PBS and brains flash frozen. Next, we isolated nuclei from dissociated brain, labeled total nuclei with a violet-fluorescing dye, and then analyzed nuclei from each group from mPlum fluorescence (FIG. 2b). All groups had nuclei, as observed by the violet dye stain. However, only the AAV-Floxed-STOP-H2B-mPlum+ AAV-Cre group showed many fluorescent mPlum+ nuclei, which demonstrates the specificity and functionality of the two-vector system (FIG. 2c). The flow data was confirmed in brain sections from the different injected groups.

### Example 4. Round 2 of Selection Enriches for Capsids that are Maintained in the NHP Spinal Cord after Intrathecal Injection

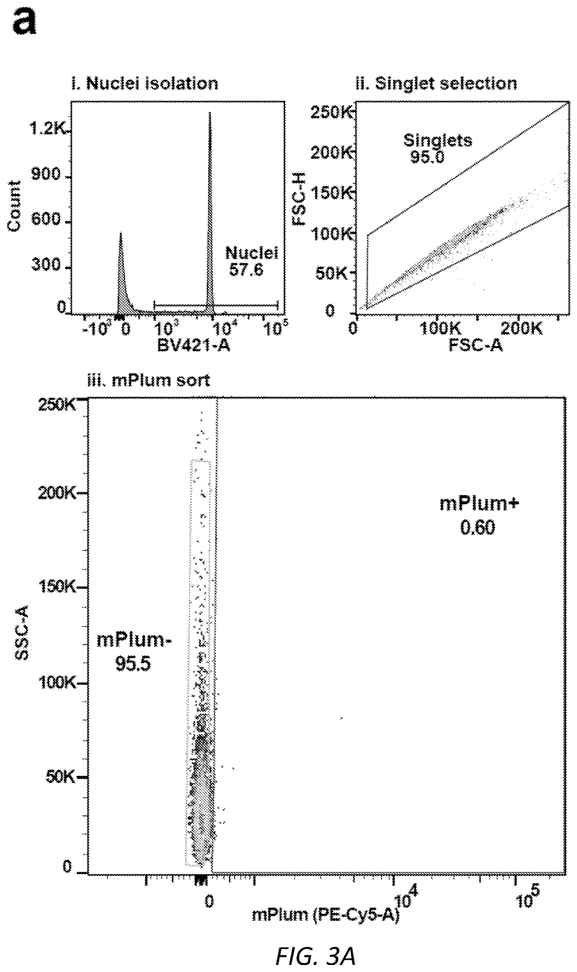

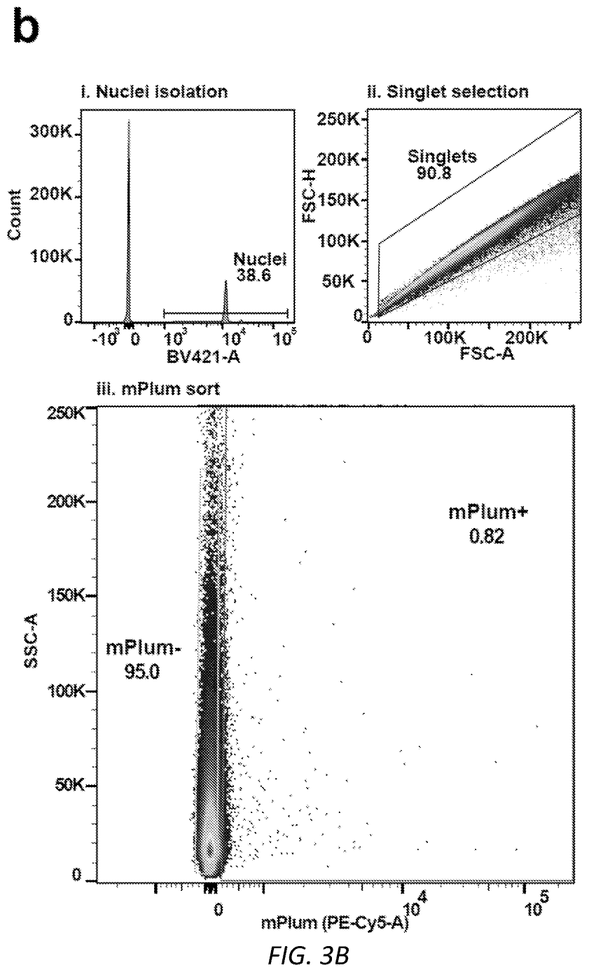

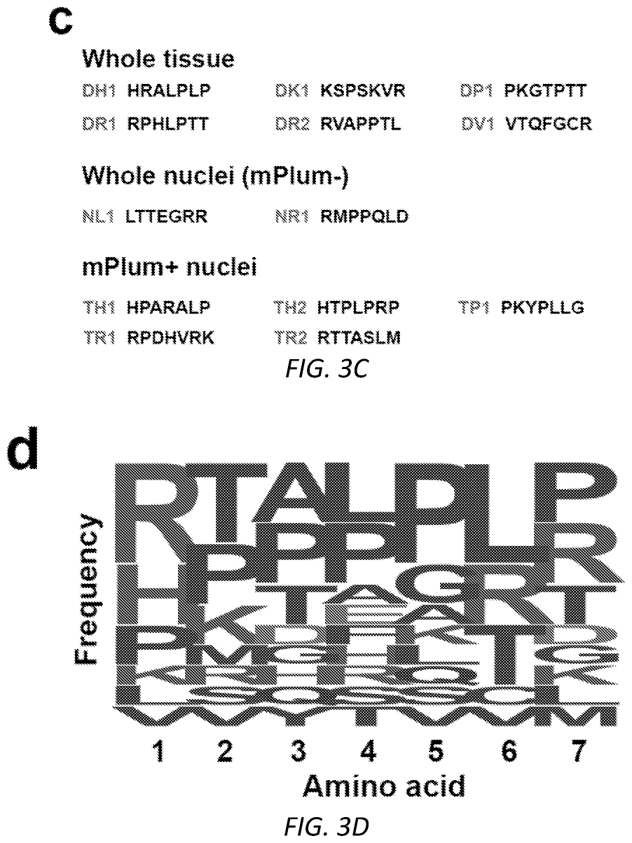

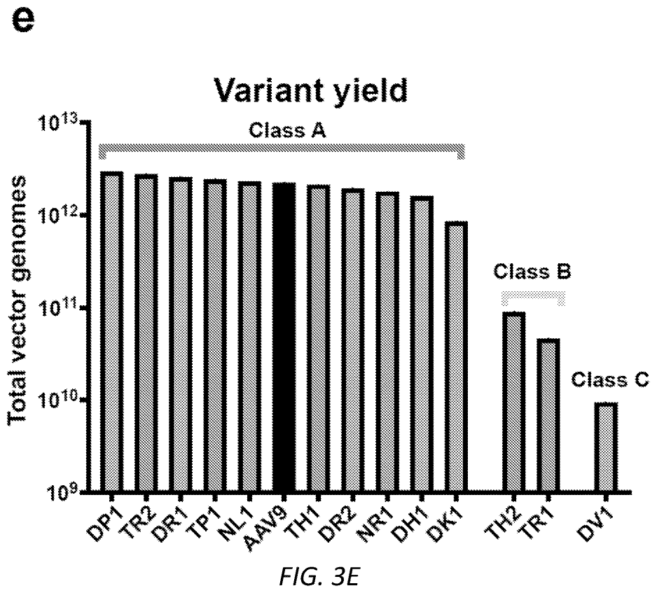

For the second round of selection, we performed two separate intrathecal injections into a single female cynomolgus macaque. The animal was first intrathecally injected with 2×10¹¹ vg of the recovered iTransduce library from round one and three hours later, while the animal was still anesthetized, intrathecally injected with 3×10¹³ vg of AAV9-CBA-Floxed-STOP-H2B-mPlum. Three weeks later the animal was killed, and spinal cord and other tissues collected and flash frozen. As shown in FIG. 1b we isolated AAV genomes containing the 21 bp inserts via three methods: (1) whole tissue as in round 1, (2) sorting dye-labeled nuclei from dissociated spinal cord, and (3) sorting H2B-mPlum positive nuclei from cells co-transduced by a transduction competent AAV capsid (Cre-expressing) and the AAV9-Floxed-STOP-H2B-mPlum vector. For whole tissue isolated DNA, we used samples from all three regions of the spinal cord as in round 1. For the nuclei isolation, we used the lumbar region of spinal cord to increase the chances of detecting mPlum expression as this was the injection region of vector and should have the highest vector concentration. We purified nuclei using iodixanol density gradients, labeled the nuclei using violet dye and flow sorted the nuclei in both mPlum negative and mPlum positive fractions. We set the mPlum+ nuclei gate based on a round 1 spinal cord sample which was not injected with the AAV9-Floxed-STOP-H2B-mPlum vector (FIG. 3a). mPlum+ nuclei were detected in the co-injected animal (FIG. 3b) and we obtained 2.09×10⁶ mPlum− nuclei and 1.70×10⁴ mPlum+ nuclei. For the whole tissue isolated DNA, and the nuclei from both mPlum+ and mPlum− fractions, we were able to amplify the insert-containing PCR product. All PCR amplicons were analyzed by NGS. For the whole tissue DNA samples isolated from thoracic and lumbar spinal cord regions we observed many of the same top peptides from round 1. There was strong enrichment however, with top peptides showing up to 15-fold to 21-fold enrichment compared to the round 1 recovered library (Tables VIA-C). The read frequency of the top clones was 0.25% and 0.28% for lumbar and thoracic, respectively. For the cervical sample there was an even greater enrichment in round 2 recovered sequences compared to the round 1 recovered library. The top peptides were enriched by 25 to 67-fold (Tables VIA-C). For the mPlum positive and negative nuclei there were far fewer variants. The top peptide in the mPlum+ fraction, HTPLPRP (SEQ ID NO: 16), represented 88% of reads and the mPlum negative was 17% of reads. Due to the surprising level of read frequency of one peptide in the mPlum positive nuclei fractions, we repeated the PCR on the nuclei DNA template for a total of three times. Interestingly in the two repeat runs of the mPlum+ fraction, the top peptides, PKYPLLG (TP1) (SEQ ID NO:14) and RPDHVRK (TR1) (SEQ ID NO:15), were both at high frequencies (>99% of reads). Based on these findings of different peptides dominating the NGS reads in the same spinal cord sample but in independent PCR reactions, we assumed that we were observing stochastic effects of the PCR owing to the low amount of nuclei present for template in the PCR. However, we considered all of these top peptides as viable candidates as they were found in the mPlum+ fraction.

To choose candidates for further screening, we included the following parameters: (1) For the whole tissue isolated DNA data, we chose peptides that showed up in all three regions of spinal cord to identify capsids that mediate good biodistribution. (2) We included peptides that were enriched in the cervical region of the spinal cord to increase chances of having capsids that can traffic from lumbar to cervical region. (3) We included candidates from the nuclei sorted samples (mPlum positive and negative) to increase chances of having transduction competent capsids. (4) We looked for common motifs in the peptides. Based on these parameters we chose 13 candidate peptides for further analysis (FIG. 3c and Table III). An amino acid frequency calculator showed a characteristic LPLP motif at positions 4-7 (FIG. 3d). We next tested the yield of individually produced candidates (in addition to AAV9 for comparison) to understand if they would be manufacturable for further development. The majority of capsids (10/13) produced very well, better than or very close to the yield of AAV9 (FIG. 3e). Three other capsids produce at lower levels. We classed these capsids A, B, and C, based on their production efficiency, with A having the highest and C the lowest (FIG. 3e).

### Example 5. Barcoded Capsid Screen Reveals Variants with Enhanced Biodistribution in Spinal Cord and Lowered Liver Biodistribution Compared to AAV9

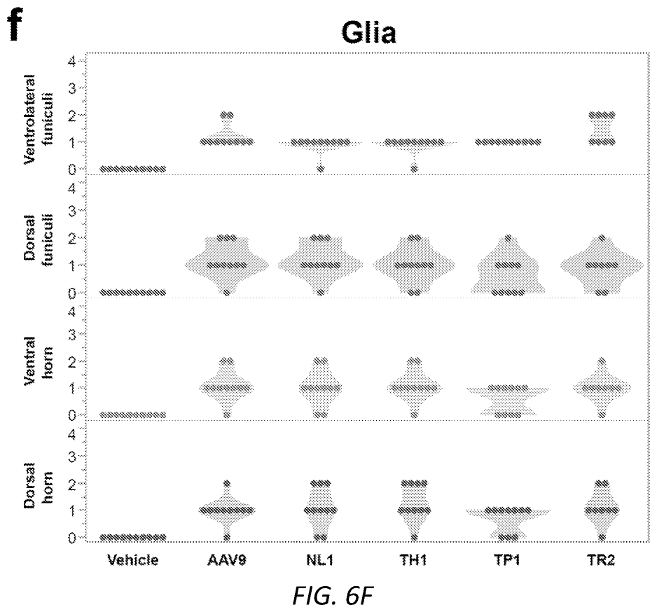

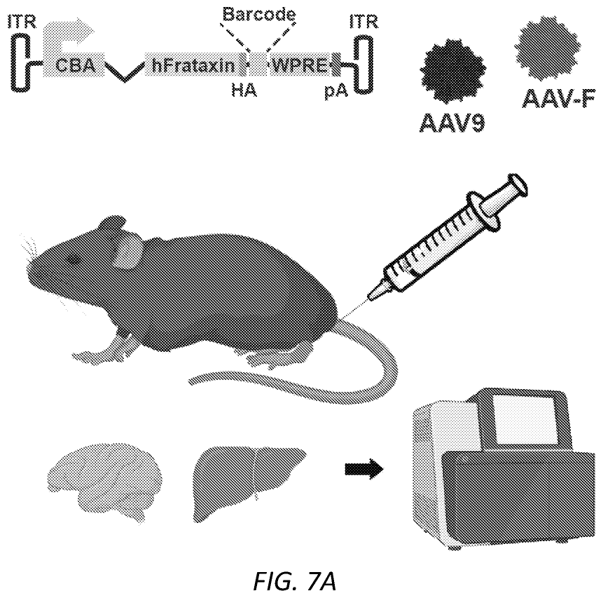

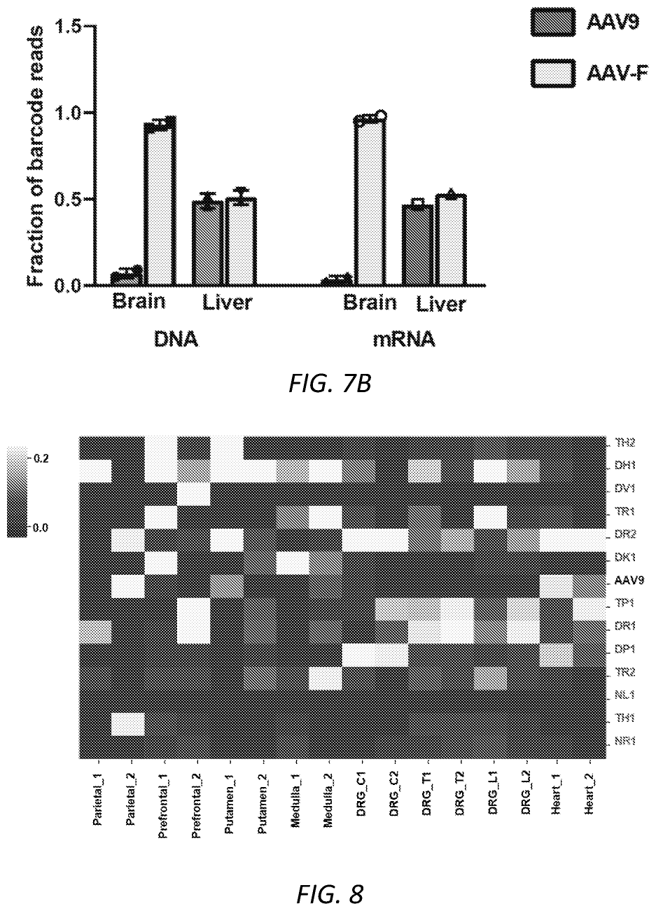

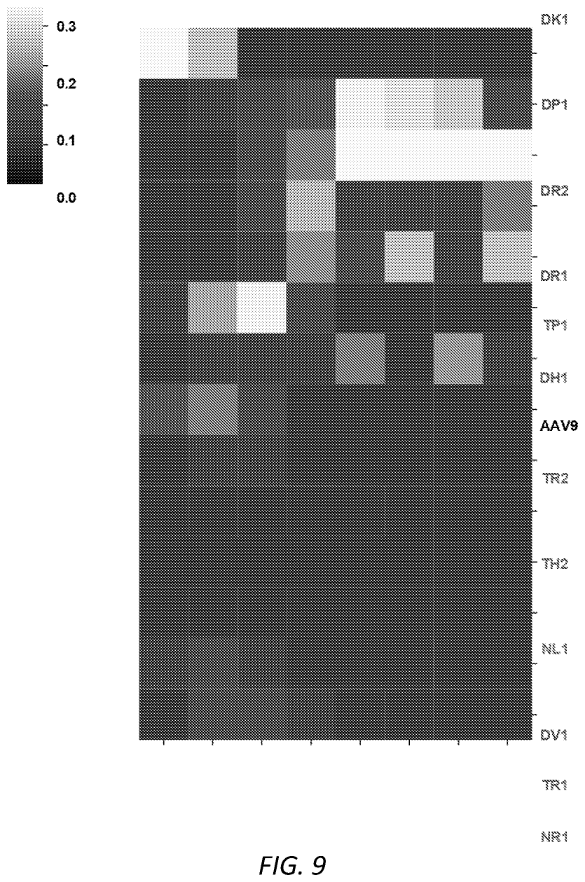

To assess the potential of the candidate peptides identified from round 2 of selection to mediate transduction of spinal cord, we engineered an AAV transgene expression cassette shown in FIG. 4a. We used a human frataxin cDNA fused to a hemagglutinin tag similar to Goertsen et al¹⁸ as this construct has shown to express well in NHP CNS and potentially avoid confounding effects of immunogenic/toxic fluorescent proteins like GFP. We added a barcode region which allows us to differentiate each of our candidate capsids via NGS at both the DNA and RNA levels. We tested the barcode system to measure biodistribution and transduction differences between AAV capsids by comparing NGS reads from barcodes at the DNA and RNA levels using systemically injected pooled AAV9 and AAV-F¹⁶ vectors (each with their own barcode) in C57BL/6 mice (FIG. 7a). Based on our prior work, the AAV-F capsid has been shown to mediate enhanced biodistribution and transduction of brain compared to AAV9, so we expected the barcode data to yield a similar profile if it was performing as expected. Similar to our prior data with single capsid comparisons¹⁶, we observed AAV-F to mediate a 13-fold increase and 28-fold increase in AAV genomes and encoded RNA, respectively (FIG. 7b). For the experiment in NHPs, we individually produced each of the 13 capsid candidates as well as AAV9, which served as our benchmarking capsid (FIG. 4a). Next, we pooled the library candidates as well as AAV9. Ten Class A capsids (which includes AAV9) were at a dose of 4×10¹¹ vg/capsid, two from Class B at a dose of 2×10¹⁰ vg/capsid, and one from Class C at 3.98×10⁹ vg. Two adult male cynomolgus monkeys (Table I) were each injected IT with 4.48×10¹² vg of the pooled barcoded capsids. Three weeks later, animals were killed, and spinal cord and other tissues isolated. We isolated DNA and RNA from the cervical, thoracic, and lumbar regions of the spinal cord and DNA and RNA from the liver. To understand the level of the pooled capsid genome in the spinal cord, we performed qPCR which detects AAV genomes using primers and probes to the poly A signal of the expression cassette. For both NHPs, AAV genomes were highest in the lumbar region with a steep gradient from lumbar to thoracic and further drop-off to cervical spinal cord (FIG. 4b), which is expected given the low dose of administered vectors and that it was a pool of different capsids. To understand the frequency of each capsid at each level of the spinal cord as well as from the liver, NGS was performed using the barcode unique for each capsid. As can be observed in the heat map of the NGS data, many capsids displayed higher frequency than AAV9 in all three spinal cord regions, with some variability between animals (FIG. 4c). Remarkably, 10 capsids outperformed AAV9 in levels of vector genomes in the lumbar region, ranging from 2 to 265-fold (FIG. 4d). Several capsids had higher levels of genomes in thoracic (up to 30-fold) and cervical (up to 10-fold) regions compared to AAV9, although there was more inter-animal variability compared to the lumbar region (FIGS. 4d and 4f). Notably, biodistribution to the liver was reduced for the majority of tested capsids (FIG. 4c), which ranged from 2- to 1,250-fold lower than AAV9 (FIG. 4e). NGS barcode analysis of DNA from heart also suggested a lower biodistribution of most of the capsids to peripheral organs compared to AAV9 (FIG. 4g). We also performed barcode analysis on the capsids in four brain regions, dorsal root ganglia (three levels of spinal cord), and heart for both NHPs (FIG. 7). There was a high degree of variability between capsids and inter-animal differences for the same capsid in some cases. Some capsids, such as DH1, showed a much higher read frequency in most brain samples compared to AAV9. Finally, we performed barcoded frequency analysis for each of the capsids in the following nerves isolated from the two NHPs: sciatic nerve, 8^{th} cranial nerve, sural nerve, and ulnar nerve (FIG. 8). While there was variability between the two animals for some capsids, there were some trends for increased frequency in certain nerves over AAV9. For examples capsids DP1 and DR2 had high read frequencies in sural and ulnar nerves compared to AAV9, DH1 had higher levels in sciatic nerve and 8^{th} cranial nerve, and DK1 in 8^{th} cranial nerve.

### Example 6. Enhanced Transduction in Spinal Cord and Lower Transduction of Liver with Several Candidate Capsids

To assess expression levels of the capsids, we assessed levels of cDNA reverse transcribed from the frataxin-HA cDNA using RT-qPCR. The cDNA levels were detected in both animals in the lumbar region, although the levels were significantly higher (12.5-fold) in NHP #1001 than #1002 (FIG. 5a). This could be due to differences in transduction efficiency between animals, as the vector genomes were quite similar between animals (FIG. 4b). The levels of cDNA in thoracic and cervical regions were not above the negative controls, likely owing to the low injected dose of each capsid, so further analysis of mRNA/cDNA was focused on the lumbar region of spinal cord and the liver. NGS was performed on the barcode cDNA from mRNA isolated from lumbar spinal cord and liver. We obtained high numbers of reads from lumbar region of both animals and frequencies of top capsids were relatively consistent between independent PCR runs (four runs/animal) (FIG. 5b). In contrast to the DNA biodistribution data, at the RNA level far fewer capsids outperformed AAV9. None of capsids from the whole tissue DNA isolation outperformed AAV9 in at least one of the injected animals by a factor of ≥2. In contrast, 1 of 2 in the nuclei isolation did, and 3 of 5 in the mPlum+ nuclei isolation did (FIG. 5b). The average enhancement ranged from 1.8 to 2.4-fold for the four capsids over AAV9 and up to 3.5-fold in one of the two animals (FIG. 5b). Liver RNA expression appeared similar to the DNA biodistribution data; capsid variants showed expression levels 2 to 30,000-fold lower compared to AAV9 (FIG. 5c).

### Example 7. Generating a Profile of Candidate Capsid Features Allows Identification of Top Candidates for Preclinical Development

Based on the data in NHPs from biodistribution in spinal cord and liver and transduction in spinal cord as well as production efficiency, we selected the top candidate capsids to move forward with (Table IV). This allowed us to narrow 13 initial candidates down to 4 peptides, which were peptides NL1, TH1, TP1, and TR2. It is important to note that all chosen capsids were from the higher-yield class A capsids injected at the same dose as AAV9.

### Example 8. NHP-Selected Capsids Efficiently Transduce Murine Spinal Cord after Intrathecal Injection

Since the selection of the library was performed in NHPs, it is likely the transduction profile would be more efficient in this species compared to rodents. However, since most neurological models of human disease are developed in transgenic mice, it would be convenient for preclinical development if the NHP-selected capsids were also functional in mice. To assess whether the AAV capsids selected in NHP were “backwards compatible”, adult 6-8 week old C57BL/6 mice were injected intrathecally in the lumbar region individually with either AAV9, NL1, TH1, TP1, or TR2 (FIG. 6a). AAV capsids packaged a single-stranded AAV-CBA-eGFP genome. AAV9, TH1, TP1, and TR2 were dosed at 8.3×10¹⁰ vg/mouse and NL1 at 4.8×10¹⁰ vg/mouse, owing to a lower titer obtained for this capsid (n=4-5 mice/capsid). Four weeks later, we harvested spinal cords to perform quantitation of GFP concentrations for each group. Caudal and rostral spinal cord segments were homogenized and a GFP ELISA was conducted. We compared the average GFP concentrations in spinal cord for the four candidate capsids to AAV9. Interestingly, both TH1 and TR2 had 3.44 (p=0.016) and 1.83-fold (p=0.013) higher concentrations of GFP compared to AAV9, respectively (FIG. 6b.)

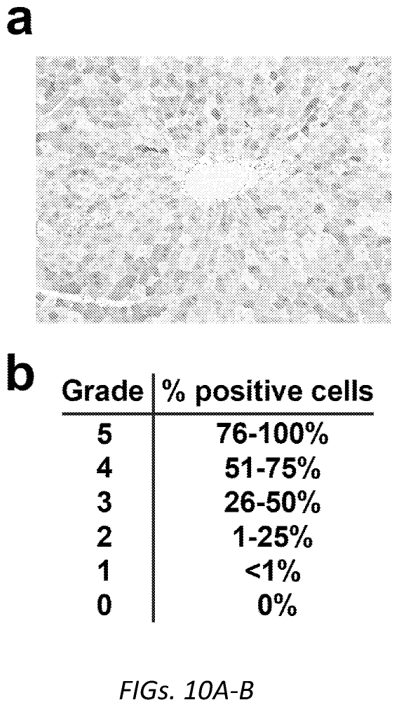

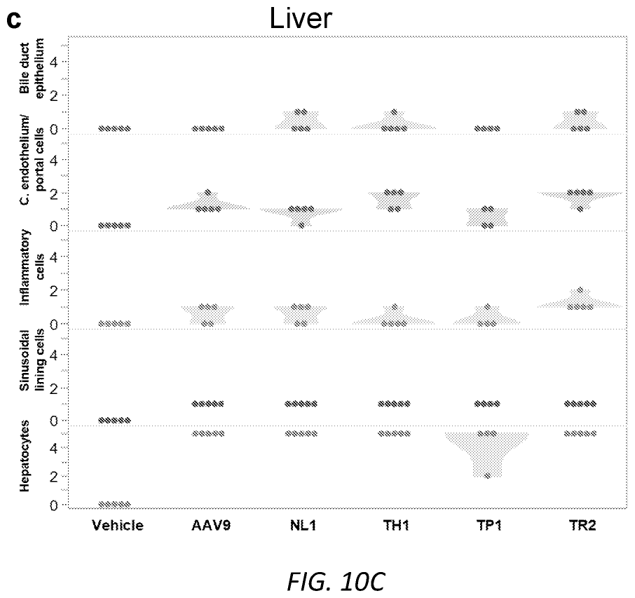

In a separate cohort of mice (n=5 mice/capsid), the same dosing scheme as above was performed. Four weeks post injection, mice were euthanized, and spinal cords and livers were harvested and cryosectioned. Spinal cords were divided into rostral and caudal regions and immunostained for GFP followed by immunohistochemical detection. Cell types were identified by morphology and scored separately for neurons, glia, pia mater cells, and ependymal cells surrounding the central canal. Representative images show transduction of neurons and glia by the two capsids that provided highest GFP levels in the spinal cord, TH1 and TR2 (FIG. 6c). A semi-quantitative data analysis was performed whereby transduction percentages were divided amongst five grading categories, with one being the lowest and five the highest percentage of GFP positive cells (FIG. 6d). Scoring for transduction percentages in both rostral and caudal spinal cord revealed that all capsids were able to transduce both neurons and glia, with some intragroup variability in grading (FIG. 6e, f). Despite being injected at 58% of the dose of the other capsids, NL1 generally trended towards higher percentages of transduced neurons in more mice/group compared to the other capsids and AAV9 (FIG. 6e, f). Glial cells were also readily transduced (FIG. 6e, f). All capsids were also able to transduce the pia mater and ependymal cells (data not shown). Livers were also examined for GFP expression based on our data in NHPs that showed lower biodistribution to this organ after intrathecal injection of the pooled candidates. Interestingly, in contrast to the NHP data, in mice the novel capsids transduced similar percentages of liver cells compared to AAV9 (FIGS. 10A-C).

Table V provides a summary of AAV vector function profiles for each peptide in non-human primates (NHPs) and mice, with levels shown calculated as fold relative to AAV9.

### Example 9. Enhanced Transduction by NHP-Selected Capsids in Human Spinal Cord Organoids

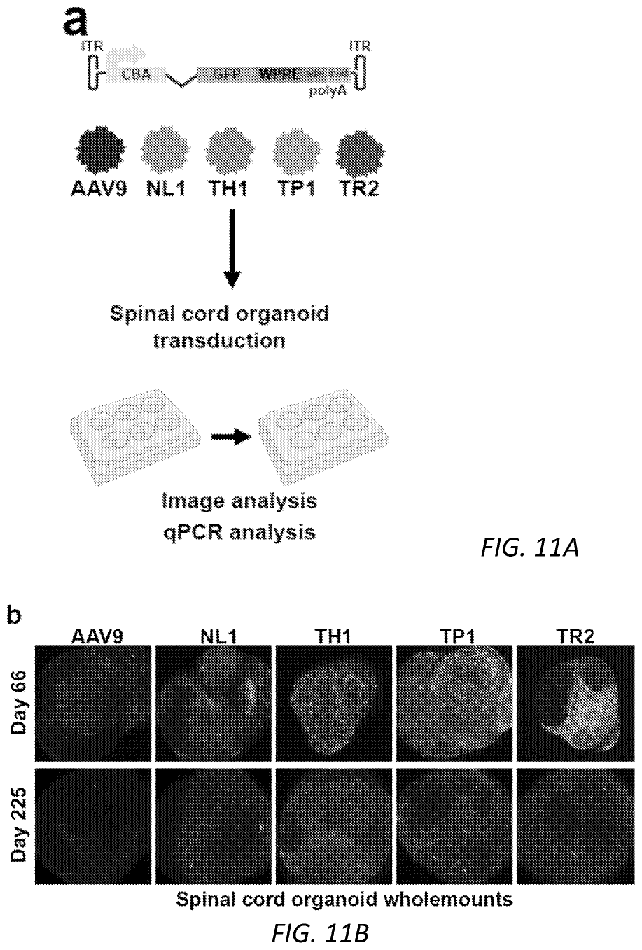

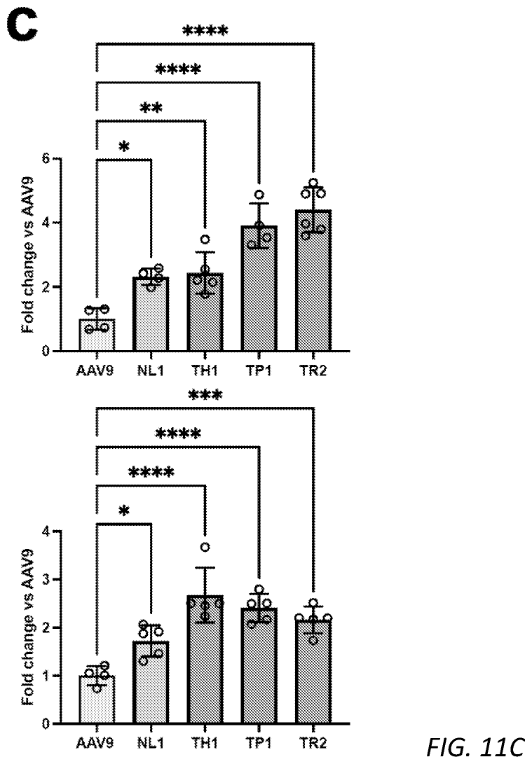

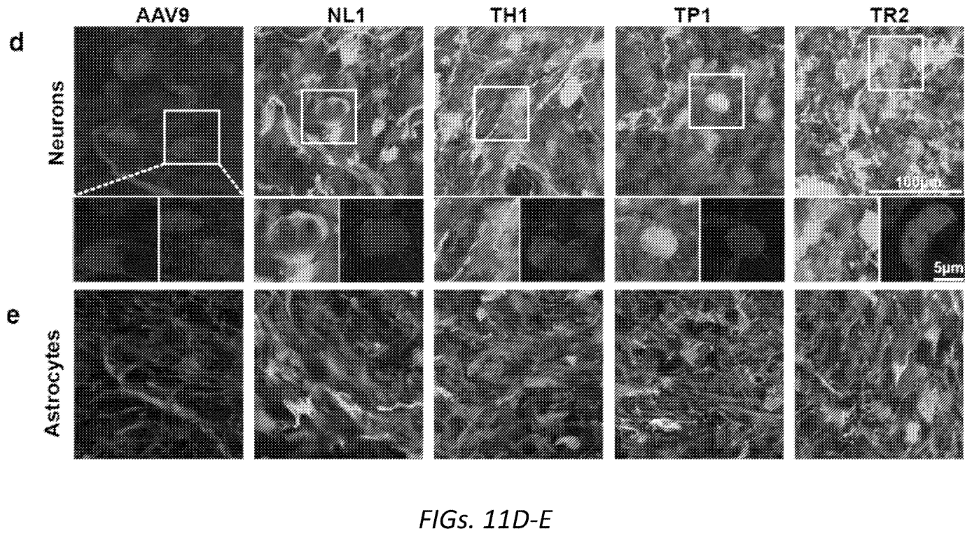

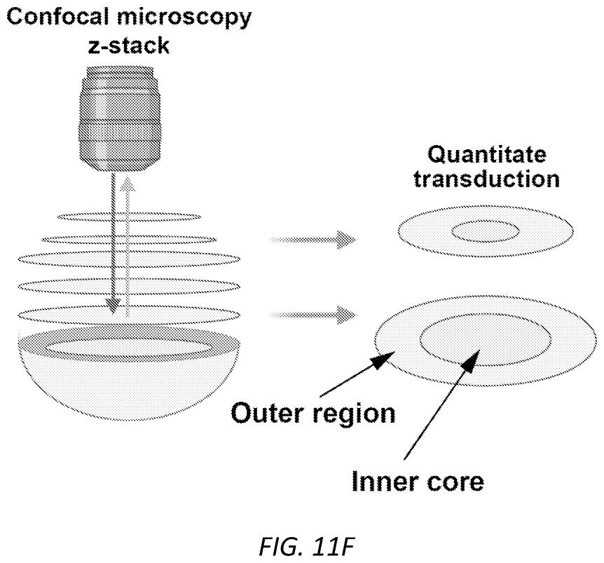

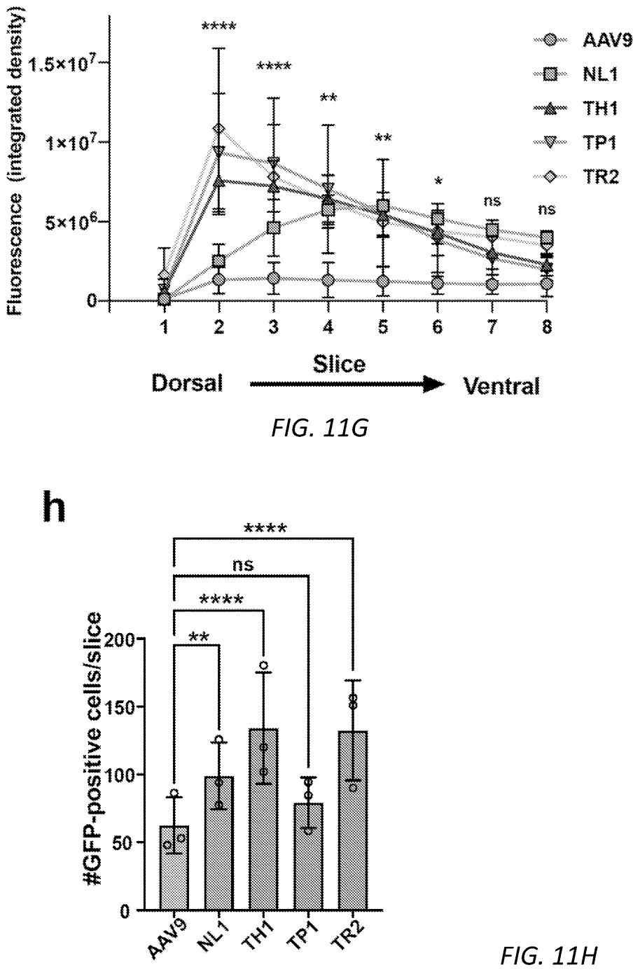

To examine the potency of transduction of the top candidate capsids in another translationally relevant model, we transduced human spinal cord organoids (SCOs) with our top four candidate capsids (NL1, TH1, TP1, and TR2) and compared them to AAV9. We tested the capsids in two separate organoid laboratories. We packaged a single-stranded AAV genome encoding a CAG-driven GFP cassette into each capsid (FIG. 11a). In SCO laboratory 1, day 66 and day 225 old SCOs were transduced with 1.17×10¹¹ vg/organoid (1.17×10¹² vg/ml) and six days later organoids were imaged for GFP expression by confocal microscopy (FIG. 11b). In day 66 old SCOs, all capsids provided statistically significant higher GFP fluorescence ranging from 2.4 to 4.4-fold higher than AAV9. Capsids TR2 and TP1 had the highest GFP fluorescence. In day 225 old SCOs, all capsids had significantly higher fluorescence as well, ranging from 1.7 to 2.7-fold higher than AAV9, with capsids TH1 and TP1 providing the highest fluorescence (FIG. 11c). Next to determine if the new capsids could transduce clinically relevant cell types in the human SCOs we performed immunostaining on transduced day 225 old organoids for the following markers: NeuN for mature neurons and GFAP for astrocytes. Both the number of GFP positive cells as well as their intensity was visibly increased for all four capsids compared to AAV9 and colocalization with astrocytes and neurons was observed (FIG. 11d, e). We also observed SOX2, a neural stem marker, colocalization and internexin, a neurite marker, colocalization with transduced cells in the day 66 and day 225 old organoids, respectively. To assess transduction throughout the organoids, z-stacks of confocal images were analyzed through multiple slices from the top (closest to the laser) to lower in the stack (FIG. 11f). Visualization through the z-stack showed obvious enhanced transduction throughout the organoids with all novel capsids compared to AAV9. We quantitated the fluorescence intensity starting at the top of the organoid (dorsal slices) and further down the image stacks (ventral slices). For all capsids except for NL1, peak fluorescence was observed in slice #2 and gradually decreased through slice #11. TR2 had the highest intensity, surpassing AAV9 by over 8-fold in slice #2 (FIG. 11g). This was likely an underestimate of enhancement as pixel values were saturated for TR2 in several of the top sections, which indicate its robust transduction capacity. Interestingly, NL1 fluorescence intensity peaked in slice #5, with a 4.9-fold increase over AAV9 (FIG. 11g). This may be due to high transduction into the core of the organoid. We also performed counts of GFP positive cells in 3 slices in each organoid with one slice near the top of the confocal stack, one in the middle, and one near the end (the same slices were analyzed for each capsid). TR2 had the highest number of GFP+ cells in the top and middle slices, being 2.9-fold and 2.1-fold higher than AAV9, respectively (FIG. 11h). TH1 had the highest GFP+ count at the bottom slice, being 2.1-fold higher than AAV9 (FIG. 11h).

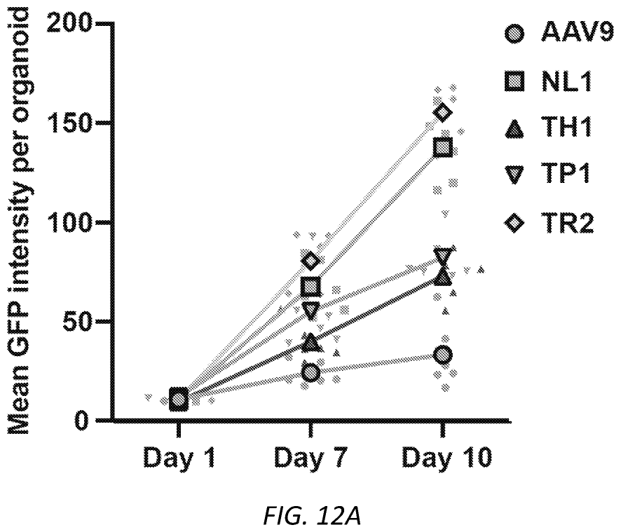

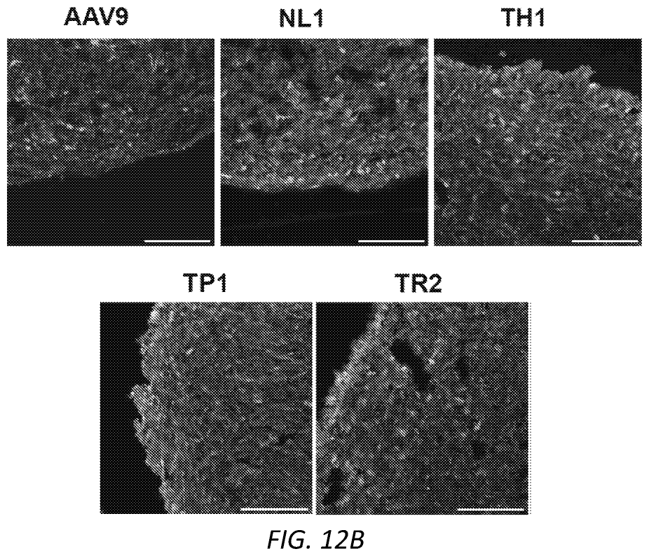

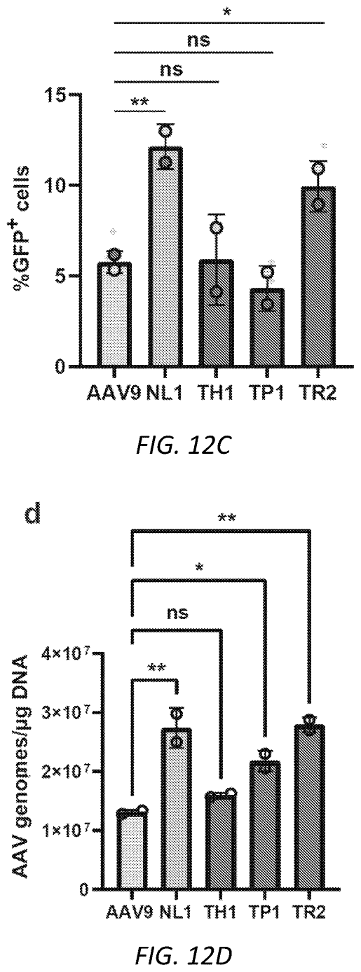

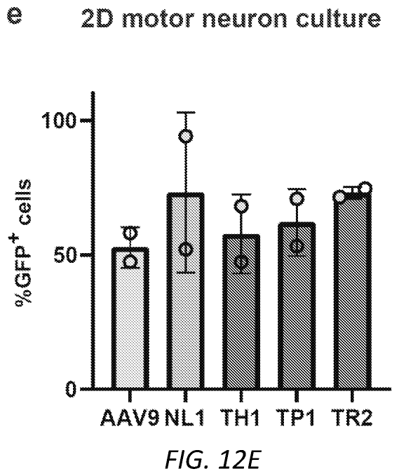

In SCO laboratory 2, day 55 old SCOs were transduced with a lower dose/concentration of each capsid, 2.5×10¹⁰ vg/organoid and 2.5×10¹⁰ vg/ml. Serial live cell imaging was performed Mean GFP fluorescence was calculated for transduced SCOs at days 1, 7, and 10 post transduction. GFP intensity increased greatly between day 1 and day 7 and was highest at day 10 for all capsids (FIGS. 12a-e). At day 10, TR2 and NL1 had the highest mean fluorescence compared to AAV9 which were 4.7-fold and 4-fold, respectively (FIG. 12a). Next, organoids were serially sectioned and imaged by confocal microscopy. Bright GFP positive cells which colocalized with NeuN were detected for all capsids (FIG. 12b). The percentage of GFP positive cells were counted in serial sections of SCOs. There was significantly enhanced (1.7 to 2.1-fold) transduction of SCOs by NL1 and TR2 compared to AAV9 (FIG. 12c). We also quantitated AAV genomes in organoids incubated with each capsid and found gene transfer to be enhanced 1.9 to 2.1-fold compared to AAV9 (FIG. 12d). Finally, we tested whether the enhanced transduction properties observed in the 3D SCOs was observed in 2D cultures of human motor neurons. Interestingly, at the tested dose, all capsids efficiently transduced these cells, with no significant enhancement compared to AAV9 (FIG. 12e).

TABLE I
NHP study information

| | Age | | | | | AAV |
|---|---|---|---|---|---|---|
| | (years, | Weight | | | | NAb |
| Animal ID | months) | (kg) | Sex | Vector | Dose (VG) | titer |
| M682 | 1 yr, 8 mo. | 2.1 | Male | Unselected library | 9.0 × 10¹¹ | <5 |
| | | | | (Round 1) | | |
| N495 | 1 yr, 11 mo. | 1.9 | Female | Round 1 recovered | 2.0 × 10¹¹ | 5 |
| | | | | library (Round 2) | | |
| MB1255 | 5 yrs, 5 mo. | 5.4 | Male | Barcoded library | 4.48 × 10¹² | <5 |
| | | | | candidates (screen) | | |
| EC1042 | 5 yrs, 2 mo. | 5.6 | Male | Barcoded library | 4.48 × 10¹² | <5 |
| | | | | candidates (screen) | | |

TABLE II
Round 1 sequencing reads of AAV capsid
inserts in spinal cord DNA.

| | | Unique sequences |
|---|---|---|
| Spinal | Unique variants | (individual |
| Cord Region | (insert reads) | 21-mer inserts) |
| Lumbar | 57,000 | 24,000 |
| Thoracic | 62,000 | 21,000 |
| Cervical | 25,000 | 1,600 |

TABLE III
Barcoded spinal cord candidate capsids for screen

| Candidate | Peptide | SEQ ID | | |
|---|---|---|---|---|
| Name | Sequence | NO: | Selection Type | Reason for choosing |
| DK1 | KSPSKVR | 4 | whole tissue | Top peptide in Cervical |
| | | | | region |
| DP1 | PKGTPTT | 5 | whole tissue | PKX motif, high |
| | | | | frequencing in lumbar |
| | | | | and thoracic regions |
| DR2 | RVAPPTL | 6 | whole tissue | Highest enrichment in |
| | | | | thoracic region, high in |
| | | | | lumbar region |
| DR1 | RPHLPTT | 7 | whole tissue | LP motif at 4,5 position, |
| | | | | RP motif, enriched in |
| | | | | cervical region |
| DH1 | HRALPLP | 8 | whole tissue | LP motif at 4,5 position |
| DV1 | VTQFGCR | 9 | whole tissue | Top peptide in Cervical |
| | | | | region |
| NL1 | LTTEGRR | 10 | nuclei | High frequency |
| NR1 | RMPPQLD | 11 | nuclei | High frequency |
| TR2 | RTTASLM | 12 | mPlum + nuclei | Detected at low |
| | | | | frequency in mPlum + |
| | | | | reads |
| TH1 | HPARALP | 13 | mPlum + nuclei | Detected at low |
| | | | | frequency in mPlum + |
| | | | | reads; similar sequence |
| | | | | to whole tissue peptide |
| | | | | HRALPLP |
| TP1 | PKYPLLG | 14 | mPlum + nuclei | PKX motif, Top |
| | | | | frequency read |
| TR1 | RPDHVRK | 15 | mPlum + nuclei | Common RP motif, Top |
| | | | | frequency read |
| TH2 | HTPLPRP | 16 | mPlum + nuclei | LP motif at 4,5; RP motif, |
| | | | | Top frequency read |

TABLE IV
Top spinal cord capsid candidate features

| | | | Spinal cord | | Liver | |
|---|---|---|---|---|---|---|
| | | SEQ | bio- | Spinal cord | biodistribution | |
| Variant | | ID | distribution | transduction | and | Production |
| name | Insert | NO: | (L-T-C) | (Lumbar) | transduction | efficiency |
| NL1 | LTTEGRR | 10 | Low | vs AAV9 | Low | High* |
| TH1 | HPARALP | 13 | High | >AAV9 | Low | High |
| TP1 | PKYPLLG | 14 | High | >AAV9 | Low | High |
| TR2 | RTTASLM | 12 | High | >AAV9 | Low | High |

*all four were similar to AAV9

TABLE V
Peptide profiles on AAV vector function in non-human primates (NHPs) and mice

| | | | | | | | Validated |
|---|---|---|---|---|---|---|---|
| | | | NHP Spinal Cord | | | Other | for |
| | | SEQ | biodistribution* | NHP lumbar | NHP liver bio- | notable NHP | transduction |
| Peptide | Peptide | ID | Cervical/Thoracic/ | spinal cord | distribution/ | tissue bio- | of mouse |
| Name | Sequence | NO: | Lumbar | transduction* | transduction* | distribution* | spinal cord |
| DK1 | KSPSKVR | 4 | 79.01 \| 2.58 \| 8.66 | 0.425 | 0.001 \| 0.0007 | 8^{th} cranial | NA |
| | | | | | | nerve: + | |
| DP1 | PKGTPTT | 5 | 2.49 \| 1.77 \| 0.00 | 1.492 | 0.700 \| 0.6190 | Brain: + | NA |
| DR2 | RVAPPTL | 6 | 11.48 \| 5.08 \| 0.59 | 1.348 | 0.481 \| 0.4826 | Heart: +; | NA |
| | | | | | | sural nerve, | |
| | | | | | | ulnar nerve: | |
| | | | | | | +; sciatic | |
| | | | | | | nerve: + | |
| DR1 | RPHLPTT | 7 | 60.71 \| 13.11 \| 0.11 | 1.400 | 0.090 \| 0.0746 | | NA |
| DH1 | HRALPLP | 8 | 170.06 \| 17.34 \| 1.30 | 1.053 | 0.001 \| 0.0007 | Brain: +; | NA |
| | | | | | | sciatic nerve: | |
| | | | | | | + | |
| DV1 | VTQFGCR | 9 | 1.45 \| 0.00 \| 0.00 | 0.000 | 0.000 \| 0.0000 | | NA |
| NL1 | LTTEGRR | 10 | 0.22 \| 0.06 \| 0.00 | 2.715 | 0.581 \| 0.5129 | | Yes |
| NR1 | RMPPQLD | 11 | 93.78 \| 7.32 \| 0.62 | 0.310 | 0.006 \| 0.0060 | | NA |
| TR2 | RTTASLM | 12 | 124.20 \| 11.02 \| 1.83 | 2.922 | 0.005 \| 0.0038 | | Yes |
| TH1 | HPARALP | 13 | 38.52 \| 7.09 \| 1.61 | 2.537 | 0.004 \| 0.0026 | | Yes |
| TP1 | PKYPLLG | 14 | 53.86 \| 11.85 \| 0.37 | 2.902 | 0.084 \| 0.0790 | DRG: + | Yes |
| TR1 | RPDHVRK | 15 | 47.54 \| 0.00 \| 0.56 | 0.198 | 0.000 \| 0.0001 | | NA |
| TH2 | HTPLPRP | 16 | 56.38 \| 7.77 \| 3.44 | 1.110 | 0.002 \| 0.0003 | | NA |

*Spinal cord and liver biodistribution (i.e., AAV barcoded genomes) and transduction (i.e. barcoded transgene mRNA) calculated as fold relative to AAV9. “Other NHP tissue biodistribution” is a qualitative measurement of notable tissues for certain peptides.
“+” indicate >biodistribution than AAV9.
NA = not assessed

TABLE VIA
Enrichment of Expression in Lumbar Spine

| Peptide | SEQ ID NO: | Rd. 2 | Rd. 1 | Enrichment |
|---|---|---|---|---|
| TSKQPPW | 17 | 0.0027 | 0.0002 | 15.22 |
| PPKPTYG | 18 | 0.0019 | 0.0002 | 11.62 |
| PTKPTFG | 19 | 0.0014 | 0.0001 | 11.42 |
| LPRLPDN | 20 | 0.0015 | 0.0001 | 10.76 |
| RHYHPTD | 21 | 0.0019 | 0.0002 | 10.61 |
| REDMPRY | 22 | 0.0021 | 0.0002 | 10.49 |
| RVAPPTL | 6 | 0.0019 | 0.0002 | 10.46 |
| VGLPRPI | 23 | 0.0037 | 0.0004 | 9.74 |
| RREQPAW | 24 | 0.0030 | 0.0003 | 9.40 |
| RPHLPTT | 7 | 0.0041 | 0.0004 | 9.10 |
| KPGTPWT | 25 | 0.0025 | 0.0003 | 8.84 |
| SAAPPTI | 26 | 0.0011 | 0.0001 | 8.57 |
| PRTPAPL | 27 | 0.0011 | 0.0001 | 8.57 |
| KHGLPTS | 28 | 0.0015 | 0.0002 | 8.46 |
| PQYLPTS | 29 | 0.0014 | 0.0002 | 7.84 |
| PLRPLAG | 30 | 0.0014 | 0.0002 | 7.69 |
| ERHPKTM | 31 | 0.0033 | 0.0004 | 7.63 |
| PLAPPTD | 32 | 0.0011 | 0.0001 | 7.50 |
| LPFPQPN | 33 | 0.0009 | 0.0001 | 7.47 |
| RNVIGVD | 34 | 0.0023 | 0.0003 | 7.42 |

TABLE VIB
Enrichment of Expression in Thoracic Spine

| Peptide | SEQ ID NO: | Rd. 2 | Rd. 1 | Enrichment |
|---|---|---|---|---|
| RVAPPTL | 6 | 0.0038 | 0.0002 | 21.10 |
| PSQGPID | 35 | 0.0021 | 0.0001 | 19.89 |
| PASLPMT | 36 | 0.0027 | 0.0001 | 18.92 |
| PPKPLMG | 37 | 0.0038 | 0.0002 | 17.76 |
| RAHLPTS | 38 | 0.0018 | 0.0001 | 16.34 |
| TSKQPPW | 17 | 0.0027 | 0.0002 | 14.92 |
| PALPSPY | 39 | 0.0020 | 0.0001 | 14.12 |
| LPFPQPN | 33 | 0.0017 | 0.0001 | 13.70 |
| IPRSPSN | 40 | 0.0028 | 0.0002 | 13.14 |
| PLRPLAG | 30 | 0.0021 | 0.0002 | 11.94 |
| TPKLPLN | 41 | 0.0019 | 0.0002 | 11.84 |
| PTRPLEG | 42 | 0.0013 | 0.0001 | 11.72 |
| QSPPPTT | 43 | 0.0012 | 0.0001 | 11.37 |
| PAFPPGP | 44 | 0.0012 | 0.0001 | 11.37 |
| LPRLPDN | 20 | 0.0016 | 0.0001 | 10.92 |
| VPRMPMS | 45 | 0.0011 | 0.0001 | 10.66 |
| SVPPPRD | 46 | 0.0015 | 0.0001 | 10.39 |
| PLRAPPL | 47 | 0.0026 | 0.0003 | 10.20 |
| TPSTPFL | 48 | 0.0011 | 0.0001 | 9.95 |
| KPNTPML | 49 | 0.0042 | 0.0004 | 9.86 |

TABLE VIC
Enrichment of Expression in Cervical Spine

| Peptide | SEQ ID NO: | Rd. 2 | Rd. 1 | Enrichment |
|---|---|---|---|---|
| PPRHPPA | 50 | 0.0072 | 0.0001 | 67.27 |
| HPSFYMI | 51 | 0.0068 | 0.0001 | 63.07 |
| THDRFCL | 52 | 0.0100 | 0.0002 | 61.67 |
| MTLCTFK | 53 | 0.0077 | 0.0001 | 61.27 |
| RVNVPVS | 54 | 0.0095 | 0.0002 | 58.86 |
| PALPANL | 55 | 0.0059 | 0.0001 | 54.66 |
| CLHPRTA | 56 | 0.0072 | 0.0001 | 50.46 |
| DKFATRT | 57 | 0.0063 | 0.0001 | 50.46 |
| PSVPLEG | 58 | 0.0091 | 0.0002 | 45.87 |
| PASRPAM | 59 | 0.0054 | 0.0001 | 43.25 |
| GRPPMNG | 60 | 0.0072 | 0.0002 | 36.69 |
| CRAIPQR | 61 | 0.0095 | 0.0003 | 35.32 |
| HKLPQPY | 62 | 0.0045 | 0.0001 | 31.53 |
| AYPCPSR | 63 | 0.0077 | 0.0003 | 30.63 |
| HTAPPAD | 64 | 0.0041 | 0.0001 | 28.38 |
| ETRPVEQ | 65 | 0.0041 | 0.0001 | 28.38 |
| KSPSKVR | 4 | 0.0122 | 0.0005 | 26.20 |
| GINARCN | 66 | 0.0027 | 0.0001 | 25.23 |
| GPRTPTI | 67 | 0.0036 | 0.0001 | 25.23 |
| NIACLAH | 68 | 0.0032 | 0.0001 | 25.23 |

### REFERENCES

1. Kuzmin, D. A., M. V. Shutova, N. R. Johnston, O. P. Smith, V. V. Fedorin, Y. S. Kukushkin, J. C. M. van der Loo, and E. C. Johnstone, The clinical landscape for AAV gene therapies. Nat Rev Drug Discov, 2021. 20(3): p. 173-174.
2. Day, J. W., R. S. Finkel, C. A. Chiriboga, A. M. Connolly, T. O. Crawford, B. T. Darras, S. T. Iannaccone, N. L. Kuntz, L. D. M. Pena, P. B. Shieh, E. C. Smith, J. M. Kwon, C. M. Zaidman, M. Schultz, D. E. Feltner, S. Tauscher-Wisniewski, H. Ouyang, D. H. Chand, D. M. Sproule, T. A. Macek, et al., Onasemnogene abeparvovec gene therapy for symptomatic infantile-onset spinal muscular atrophy in patients with two copies of SMN2 (STRIVE): an open-label, single-arm, multicentre, phase 3 trial. Lancet Neurol, 2021. 20(4): p. 284-293.
3. Thomsen, G., A. H. M. Burghes, C. Hsieh, J. Do, B. T. T. Chu, S. Perry, B. Barkho, P. Kaufmann, D. M. Sproule, D. E. Feltner, W. K. Chung, V. L. McGovern, R. F. Hevner, M. Conces, C. R. Pierson, M. Scoto, F. Muntoni, J. R. Mendell, and K. D. Foust, Biodistribution of onasemnogene abeparvovec DNA, mRNA and SMN protein in human tissue. Nat Med, 2021. 27(10): p. 1701-1711.
4. Morales, L., Y. Gambhir, J. Bennett, and H. H. Stedman, Broader Implications of Progressive Liver Dysfunction and Lethal Sepsis in Two Boys following Systemic High-Dose AAV. Mol Ther, 2020. 28(8): p. 1753-1755.
5. Wilson, J. M. and T. R. Flotte, Moving Forward After Two Deaths in a Gene Therapy Trial of Myotubular Myopathy. Hum Gene Ther, 2020. 31(13-14): p. 695-696.
6. Hordeaux, J., E. L. Buza, C. Dyer, T. Goode, T. W. Mitchell, L. Richman, N. Denton, C. Hinderer, N. Katz, R. Schmid, R. Miller, G. R. Choudhury, M. Horiuchi, K. Nambiar, H. Yan, M. Li, and J. M. Wilson, Adeno-Associated Virus-Induced Dorsal Root Ganglion Pathology. Hum Gene Ther, 2020. 31(15-16): p. 808-818.
7. Hordeaux, J., E. L. Buza, B. Jeffrey, C. Song, T. Jahan, Y. Yuan, Y. Zhu, P. Bell, M. Li, J. A. Chichester, R. Calcedo, and J. M. Wilson, MicroRNA-mediated inhibition of transgene expression reduces dorsal root ganglion toxicity by AAV vectors in primates. Sci Transl Med, 2020. 12(569).
8. Bey, K., J. Deniaud, L. Dubreil, B. Joussemet, J. Cristini, C. Ciron, J. Hordeaux, M. Le Boulc'h, K. Marche, M. Maquigneau, M. Guilbaud, R. Moreau, T. Larcher, J. Y. Deschamps, M. Fusellier, V. Blouin, C. Sevin, N. Cartier, O. Adjali, P. Aubourg, et al., Intra-CSF AAV9 and AAVrh10 Administration in Nonhuman Primates: Promising Routes and Vectors for Which Neurological Diseases?Mol Ther Methods Clin Dev, 2020. 17: p. 771-784.
9. Meyer, K., L. Ferraiuolo, L. Schmelzer, L. Braun, V. McGovern, S. Likhite, O. Michels, A. Govoni, J. Fitzgerald, P. Morales, K. D. Foust, J. R. Mendell, A. H. Burghes, and B. K. Kaspar, Improving single injection CSF delivery of AAV9-mediated gene therapy for SMA: a dose-response study in mice and nonhuman primates. Mol Ther, 2015. 23(3): p. 477-87.
10. Castle, M. J., Y. Cheng, A. Asokan, and M. H. Tuszynski, Physical positioning markedly enhances brain transduction after intrathecal AAV9 infusion. Sci Adv, 2018. 4(11): p. eaau9859.
11. Bailey, R. M., A. Rozenberg, and S. J. Gray, Comparison of high-dose intracisterna magna and lumbar puncture intrathecal delivery of AAV9 in mice to treat neuropathies. Brain Res, 2020. 1739: p. 146832.
12. Meseck, E. K., G. Guibinga, S. Wang, C. McElroy, E. Hudry, and K. Mansfield, Intrathecal sc-AAV9-CB-GFP: Systemic Distribution Predominates Following Single-Dose Administration in Cynomolgus Macaques. Toxicol Pathol, 2022. 50(4): p. 415-431.
13. Ballon, D. J., J. B. Rosenberg, E. K. Fung, A. Nikolopoulou, P. Kothari, B. P. De, B. He, A. Chen, L. A. Heier, D. Sondhi, S. M. Kaminsky, P. D. Mozley, J. W. Babich, and R. G. Crystal, Quantitative Whole-Body Imaging of I-124-Labeled Adeno-Associated Viral Vector Biodistribution in Nonhuman Primates. Hum Gene Ther, 2020. 31(23-24): p. 1237-1259.
14. Hudry, E., F. Aihara, E. Meseck, K. Mansfield, C. McElroy, D. Chand, F. F. Tukov, and K. Penraat, Liver Injury in Cynomolgus Monkeys Following Intravenous and Intrathecal scAAV9 Gene Therapy Delivery. Mol Ther, 2023.
15. Finkel, R. S., B. T. Darras, J. R. Mendell, J. W. Day, N. L. Kuntz, A. M. Connolly, C. M. Zaidman, T. O. Crawford, R. J. Butterfield, P. B. Shieh, G. Tennekoon, J. F. Brandsema, S. T. Iannaccone, J. Shoffner, S. Kavanagh, T. A. Macek, and S. Tauscher-Wisniewski, Intrathecal Onasemnogene Abeparvovec for Sitting, Nonambulatory Patients with Spinal Muscular Atrophy: Phase I Ascending-Dose Study (STRONG). J Neuromuscul Dis, 2023. 10(3): p. 389-404.
16. Hanlon, K. S., J. C. Meltzer, T. Buzhdygan, M. J. Cheng, M. Sena-Esteves, R. E. Bennett, T. P. Sullivan, R. Razmpour, Y. Gong, C. Ng, J. Nammour, D. Maiz, S. Dujardin, S. H. Ramirez, E. Hudry, and C. A. Maguire, Selection of an Efficient AAV Vector for Robust CNS Transgene Expression. Mol Ther Methods Clin Dev, 2019. 15: p. 320-332.
17. Huang, Q., A. T. Chen, K. Y. Chan, H. Sorensen, A. J. Barry, B. Azari, T. Beddow, Q. Zheng, B. Zhao, I. G. Tobey, F.-E. Eid, Y. A. Chan, and B. E. Deverman, Targeting AAV vectors to the CNS via <em>de novo</em>engineered capsid-receptor interactions. bioRxiv, 2022: p. 2022.10.31.514553.
18. Goertsen, D., N. C. Flytzanis, N. Goeden, M. R. Chuapoco, A. Cummins, Y. Chen, Y. Fan, Q. Zhang, J. Sharma, Y. Duan, L. Wang, G. Feng, Y. Chen, N.Y. Ip, J. Pickel, and V. Gradinaru, AAV capsid variants with brain-wide transgene expression and decreased liver targeting after intravenous delivery in mouse and marmoset. Nat Neurosci, 2022. 25(1): p. 106-115.
19. Lek, A., B. Wong, A. Keeler, M. Blackwood, K. Ma, S. Huang, K. Sylvia, A. R. Batista, R. Artinian, D. Kokoski, S. Parajuli, J. Putra, C. K. Carreon, H. Lidov, K. Woodman, S. Pajusalu, J. M. Spinazzola, T. Gallagher, J. LaRovere, D. Baulderson, et al., Unexpected Death of a Duchenne Muscular Dystrophy Patient in an N-of-1 Trial of rAAV9-delivered CRISPR-transactivator. medRxiv, 2023: p. 2023.05.16.23289881.
20. Gray, S. J., S. Nagabhushan Kalburgi, T. J. McCown, and R. Jude Samulski, Global CNS gene delivery and evasion of anti-AAV-neutralizing antibodies by intrathecal AAV administration in non-human primates. Gene Ther, 2013. 20(4): p. 450-9.
21. Finneran, D. J., I. P. Njoku, D. Flores-Pazarin, M. R. Ranabothu, K. R. Nash, D. Morgan, and M. N. Gordon, Toward Development of Neuron Specific Transduction After Systemic Delivery of Viral Vectors. Front Neurol, 2021. 12: p. 685802.
22. Dhungel, B., C. A. Ramlogan-Steel, and J. C. Steel, Synergistic and independent action of endogenous microRNAs 122a and 199a for post-transcriptional liver detargeting of gene vectors. Sci Rep, 2018. 8(1): p. 15539.
23. Ahmed, S. S., H. Li, C. Cao, E. M. Sikoglu, A. R. Denninger, Q. Su, S. Eaton, A. A. Liso Navarro, J. Xie, S. Szucs, H. Zhang, C. Moore, D. A. Kirschner, T. N. Seyfried, T. R. Flotte, R. Matalon, and G. Gao, A single intravenous rAAV injection as late as P20 achieves efficacious and sustained CNS Gene therapy in Canavan mice. Mol Ther, 2013. 21(12): p. 2136-47.
24. Nonnenmacher, M., W. Wang, M. A. Child, X. Q. Ren, C. Huang, A. Z. Ren, J. Tocci, Q. Chen, K. Bittner, K. Tyson, N. Pande, C. H. Chung, S. M. Paul, and J. Hou, Rapid evolution of blood-brain-barrier-penetrating AAV capsids by RNA-driven biopanning. Mol Ther Methods Clin Dev, 2021. 20: p. 366-378.
25. Korbelin, J., G. Dogbevia, S. Michelfelder, D. A. Ridder, A. Hunger, J. Wenzel, H. Seismann, M. Lampe, J. Bannach, M. Pasparakis, J. A. Kleinschmidt, M. Schwaninger, and M. Trepel, A brain microvasculature endothelial cell-specific viral vector with the potential to treat neurovascular and neurological diseases. EMBO Mol Med, 2016. 8(6): p. 609-25.
26. Deverman, B. E., P. L. Pravdo, B. P. Simpson, S. R. Kumar, K. Y. Chan, A. Banerjee, W. L. Wu, B. Yang, N. Huber, S. P. Pasca, and V. Gradinaru, Cre-dependent selection yields AAV variants for widespread gene transfer to the adult brain. Nat Biotechnol, 2016. 34(2): p. 204-9.
27. Penzes, J. J., P. Chipman, N. Bhattacharya, A. Zeher, R. Huang, R. McKenna, and M. Agbandje-McKenna, Adeno-associated Virus 9 Structural Rearrangements Induced by Endosomal Trafficking pH and Glycan Attachment. J Virol, 2021. 95(19): p. e0084321.
28. Maguire, C. A., L. Balaj, S. Sivaraman, M. H. Crommentuijn, M. Ericsson, L. Mincheva-Nilsson, V. Baranov, D. Gianni, B. A. Tannous, M. Sena-Esteves, X. O. Breakefield, and J. Skog, Microvesicle-associated AAV vector as a novel gene delivery system. Mol Ther, 2012. 20(5): p. 960-71.
29. Crooks, G. E., G. Hon, J. M. Chandonia, and S. E. Brenner, WebLogo: a sequence logo generator. Genome Res, 2004. 14(6): p. 1188-90.
30. Schneider, T. D. and R. M. Stephens, Sequence logos: a new way to display consensus sequences. Nucleic Acids Res, 1990. 18(20): p. 6097-100.
31. Seo, K., S. Cho, H. Shin, A. Shin, J. H. Lee, J. H. Kim, B. Lee, H. Jang, Y. Kim, H. M. Cho, Y. Park, H. Y. Kim, T. Lee, W. Y. Park, Y. J. Kim, E. Yang, D. Geum, H. Kim, I. J. Cho, S. Lee, et al., Symmetry Breaking of Human Pluripotent Stem Cells (hPSCs) in Micropattern Generates a Polarized Spinal Cord-Like Organoid (pSCO) with Dorsoventral Organization. Adv Sci (Weinh), 2023: p. e2301787.
32. Salabarria, S. M., M. Corti, K. E. Coleman, M. B. Wichman, J. A. Berthy, P. D'Souza, C. J. Tifft, R. W. Herzog, M. E. Elder, L. R. Shoemaker, C. Leon-Astudillo, F. Tavakkoli, D. H. Kirn, J. D. Schwartz, and B. J. Byrne, Thrombotic microangiopathy following systemic AAV administration is dependent on anti-capsid antibodies. J Clin Invest, 2024. 134(1).
33. Depla, J. A., M. Sogorb-Gonzalez, L. A. Mulder, V. M. Heine, P. Konstantinova, S. J. van Deventer, K. C. Wolthers, D. Pajkrt, A. Sridhar, and M. M. Evers, Cerebral Organoids: A Human Model for AAV Capsid Selection and Therapeutic Transgene Efficacy in the Brain. Mol Ther Methods Clin Dev, 2020. 18: p. 167-175.
34. Ramamurthy, R. M., A. Atala, C. D. Porada, and G. Almeida-Porada, Organoids and microphysiological systems: Promising models for accelerating AAV gene therapy studies. Front Immunol, 2022. 13: p. 1011143.
35. Garita-Hernandez, M., F. Routet, L. Guibbal, H. Khabou, L. Toualbi, L. Riancho, S. Reichman, J. Duebel, J. A. Sahel, O. Goureau, and D. Dalkara, AAV-Mediated Gene Delivery to 3D Retinal Organoids Derived from Human Induced Pluripotent Stem Cells. Int J Mol Sci, 2020. 21(3).
36. Loewa, A., J. J. Feng, and S. Hedtrich, Human disease models in drug development. Nat Rev Bioeng, 2023: p. 1-15.
37. Haase, R., L. A. Royer, P. Steinbach, D. Schmidt, A. Dibrov, U. Schmidt, M. Weigert, N. Maghelli, P. Tomancak, F. Jug, and E. W. Myers, CLIJ: GPU-accelerated image processing for everyone. Nat Methods, 2020. 17(1): p. 5-6.
38. Gyorgy, B., E. J. Meijer, M. V. Ivanchenko, K. Tenneson, F. Emond, K. S. Hanlon, A. A. Indzhykulian, A. Volak, K. D. Karavitaki, P. I. Tamvakologos, M. Vezina, V. K. Berezovskii, R. T. Born, M. O'Brien, J. F. Lafond, Y. Arsenijevic, M. A. Kenna, C. A. Maguire, and D. P. Corey, Gene Transfer with AAV9-PHP.B Rescues Hearing in a Mouse Model of Usher Syndrome 3A and Transduces Hair Cells in a Non-human Primate. Mol Ther Methods Clin Dev, 2019. 13: p. 1-13.
39. Andersen, J., O. Revah, Y. Miura, N. Thom, N. D. Amin, K. W. Kelley, M. Singh, X. Chen, M. V. Thete, E. M. Walczak, H. Vogel, H. C. Fan, and S. P. Pasca, Generation of Functional Human 3D Cortico-Motor Assembloids. Cell, 2020. 183(7): p. 1913-1929 e26.
40. Du, Z. W., H. Chen, H. Liu, J. Lu, K. Qian, C. L. Huang, X. Zhong, F. Fan, and S. C. Zhang, Generation and expansion of highly pure motor neuron progenitors from human pluripotent stem cells. Nat Commun, 2015. 6: p. 6626.
41. Al-Zaidy, S. A., et al., (2019). AVXS-101 (Onasemnogene Abeparvovec) for SMA1: Comparative Study with a Prospective Natural History Cohort. J. Neuromuscul. Dis. doi:10.3233/JND-190403.
42. Caron, N. S., et al. (2020). Potent and sustained huntingtin lowering via AAV5 encoding miRNA preserves striatal volume and cognitive function in a humanized mouse model of Huntington disease. Nucleic Acids Res. doi:10.1093/nar/gkz976.
43. Cheah, P. S., et al. (2021). Gene therapy for tuberous sclerosis complex type 2 in a mouse model by delivery of AAV9 encoding a condensed form of tuberin. Science Advances. 10.1126/sciadv.abb1703
44. Christine, C. W., et al. (2019). Magnetic resonance imaging-guided phase 1 trial of putaminal AADC gene therapy for Parkinson's disease. Ann. Neurol. doi:10.1002/ana.25450.
45. Eichler, F., et al. (2017). Hematopoietic stem-cell gene therapy for cerebral adrenoleukodystrophy. N. Engl. J. Med. doi:10.1056/NEJMoa1700554.
46. Flotte, T. R., et al. (2022). AAV gene therapy for Tay-Sachs disease. Nature Medicine. doi: 10.1038/s41591-021-01664-4
47. Gomez, K., et al. (2023). Identification and targeting of a unique NaV1.7 domain driving chronic pain. PNAS. pnas.org/doi/10.1073/pnas.2217800120
48. Gong, Y., et al. (2015). Adenoassociated virus serotype 9-mediated gene therapy for X-linked adrenoleukodystrophy. Mol. Ther. doi:10.1038/mt.2015.6.
49. Griciuc, A., et al. (2013). Alzheimer's disease risk gene cd33 inhibits microglial uptake of amyloid beta. Neuron. doi:10.1016/j.neuron.2013.04.014.
50. Griciuc, A. et al. (2020). Gene therapy for Alzheimer's disease targeting CD33 reduces amyloid beta accumulation and neuroinflammation. Human Molecular Genetics. doi: 10.1093/hmg/ddaa179.
51. Hocquemiller, M., et al. (2022). AAVrh10 vector corrects pathology in animal models of GM1 gangliosidosis and achieves widespread distribution in the CNS of nonhuman primates. Molecular Therapy—Methods and Clinical Development. DOI: doi.org/10.1016/j.omtm.2022.10.004
52. Hughes, M. P., et al. (2018). AAV9 intracerebroventricular gene therapy improves lifespan, locomotor function and pathology in a mouse model of Niemann-Pick type C1 disease. Hum. Mol. Genet. doi:10.1093/hmg/ddy212.
53. Kagiava, A., et al. (2021). Efficacy of AAV serotypes to target Schwann cells after intrathecal and intravenous delivery. Scientific Reports. doi.org/10.1038/s41598-021-02694-1
54. Keskin, S., et al. (2019). AAV5-miHTT Lowers Huntingtin mRNA and Protein without Off-Target Effects in Patient-Derived Neuronal Cultures and Astrocytes. Mol. Ther.—Methods Clin. Dev. doi:10.1016/j.omtm.2019.09.010.
55. Lahey, H. G., et al. (2020). Pronounced Therapeutic Benefit of a Single Bidirectional AAV Vector Administered Systemically in Sandhoff Mice. Molecular Therapy. DOI: 10.1016/j.ymthe.2020.06.021
56. Leone, P., et al. (2012). Long-term follow-up after gene therapy for Canavan disease. Sci. Transl. Med. doi:10.1126/scitranslmed.3003454.
57. Mueller, C., et al. (2020). SOD1 Suppression with Adeno-Associated Virus and MicroRNA in Familial ALS. New England Journal of Medicine. DOI: 10.1056/NEJMoa2005056
58. Pearson, T. S., et al. (2021). Gene therapy for aromatic L-amino acid decarboxylase deficiency by MR-guided direct delivery of AAV2-AADC to midbrain dopaminergic neurons. Nature Communications. doi.org/10.1038/s41467-021-24524-8
59. Prabhakar S., et al. (2019). Long-Term Therapeutic Efficacy of Intravenous AAV-Mediated Hamartin Replacement in Mouse Model of Tuberous Sclerosis Type 1. Molecular Therapy-Methods and Clinical Development. doi: 10.1016/j.omtm.2019.08.003.
60. Prabhakar S., et al. (2022). Gene replacement therapy in a schwannoma mouse model of neurofibromatosis type 2. Molecular Therapy-Methods and Clinical Development. doi.org/10.1016/j.omtm.2022.06.012

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An AAV capsid protein comprising an amino acid sequence that comprises at least four contiguous amino acids from the sequence TR2 (RTTASLM, SEQ ID NO:12), NL1 (LTTEGRR, SEQ ID NO:10), TH1 (HPARALP, SEQ ID NO:13), or TP1 (PKYPLLG, SEQ ID NO:14).

2. The AAV capsid protein of claim 1, comprising an amino acid sequence that comprises at least five contiguous amino acids from the sequence TR2 (RTTASLM, SEQ ID NO:12), NL1 (LTTEGRR, SEQ ID NO:10), TH1 (HPARALP, SEQ ID NO:13), or TP1 (PKYPLLG, SEQ ID NO: 14).

3. The AAV capsid protein of claim 1, comprising an amino acid sequence that comprises at least six or all seven contiguous amino acids from the sequence TR2 (RTTASLM, SEQ ID NO:12), NL1 (LTTEGRR, SEQ ID NO:10), TH1 (HPARALP, SEQ ID NO:13), or TP1 (PKYPLLG, SEQ ID NO:14).

4. The AAV capsid protein of claim 1, wherein the AAV is AAV9.

5. The AAV capsid protein of claim 1, comprising AAV9 VP1.

6. The AAV capsid protein of claim 5, wherein the sequence is inserted in a position corresponding to amino acids 588 and 589 of AAV9 VP1.

7. A nucleic acid encoding the AAV capsid protein of claim 1, optionally a recombinant episome.

8. An AAV comprising the capsid protein of claim 1, and preferably not comprising a wild type VP1, VP2, or VP3 capsid protein.

9. The AAV of claim 8, further comprising a transgene, preferably a therapeutic transgene, optionally a transgene listed in Table A.

10. A method of delivering a transgene to a cell, the method comprising contacting the cell with the AAV of claim 1.

11. The method of claim 10, wherein the cell is a neuron (optionally a dorsal root ganglion neuron or spiral ganglion neuron), astrocyte, glial cell, Schwann Cell of a peripheral nerve, or cardiomyocyte.

12. The method of claim 11, wherein the cell is in a living subject.

13. The method of claim 11, wherein the subject is a mammalian subject.

14. The method of claim 10, wherein the cell is in a tissue selected from the brain, spinal cord, dorsal root ganglion, Schwann Cell of a peripheral nerve, or heart, and a combination thereof.

15. The method of claim 14, wherein the subject has a disease listed in Table A.

16. The method of claim 10, wherein the cell is in the spinal cord of the subject, and the AAV is administered by intrathecal delivery.

17. The method of claim 16, wherein the intrathecal delivery is via lumbar injection, cisternal magna injection, or intraparenchymal injection.

18. The method of claim 10, wherein the AAV is delivered by parenteral delivery, preferably via intravenous, intraarterial, subcutaneous, intraperitoneal, or intramuscular delivery.


SEQUENCE LISTING (68 sequences)

SEQ ID NO: 1 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 20 bp, synthetic construct
  source 1..20: mol_type = other DNA
aatcctggacctgctatggc

SEQ ID NO: 3 - DNA, 20 bp, synthetic construct
  source 1..20: mol_type = other DNA
tgccaaaccatacccggaag

SEQ ID NO: 4 - protein, 7 aa, synthetic construct
KSPSKVR

SEQ ID NO: 5 - protein, 7 aa, synthetic construct
PKGTPTT

SEQ ID NO: 6 - protein, 7 aa, synthetic construct
RVAPPTL

SEQ ID NO: 7 - protein, 7 aa, synthetic construct
RPHLPTT

SEQ ID NO: 8 - protein, 7 aa, synthetic construct
HRALPLP

SEQ ID NO: 9 - protein, 7 aa, synthetic construct
VTQFGCR

SEQ ID NO: 10 - protein, 7 aa, synthetic construct
LTTEGRR

SEQ ID NO: 11 - protein, 7 aa, synthetic construct
RMPPQLD

SEQ ID NO: 12 - protein, 7 aa, synthetic construct
RTTASLM

SEQ ID NO: 13 - protein, 7 aa, synthetic construct
HPARALP

SEQ ID NO: 14 - protein, 7 aa, synthetic construct
PKYPLLG

SEQ ID NO: 15 - protein, 7 aa, synthetic construct
RPDHVRK

SEQ ID NO: 16 - protein, 7 aa, synthetic construct
HTPLPRP

SEQ ID NO: 17 - protein, 7 aa, synthetic construct
TSKQPPW

SEQ ID NO: 18 - protein, 7 aa, synthetic construct
PPKPTYG

SEQ ID NO: 19 - protein, 7 aa, synthetic construct
PTKPTFG

SEQ ID NO: 20 - protein, 7 aa, synthetic construct
LPRLPDN

SEQ ID NO: 21 - protein, 7 aa, synthetic construct
RHYHPTD

SEQ ID NO: 22 - protein, 7 aa, synthetic construct
REDMPRY

SEQ ID NO: 23 - protein, 7 aa, synthetic construct
VGLPRPI

SEQ ID NO: 24 - protein, 7 aa, synthetic construct
RREQPAW

SEQ ID NO: 25 - protein, 7 aa, synthetic construct
KPGTPWT

SEQ ID NO: 26 - protein, 7 aa, synthetic construct
SAAPPTI

SEQ ID NO: 27 - skipped

SEQ ID NO: 28 - protein, 7 aa, synthetic construct
KHGLPTS

SEQ ID NO: 29 - protein, 7 aa, synthetic construct
PQYLPTS

SEQ ID NO: 30 - protein, 7 aa, synthetic construct
PLRPLAG

SEQ ID NO: 31 - protein, 7 aa, synthetic construct
ERHPKTM

SEQ ID NO: 32 - protein, 7 aa, synthetic construct
PLAPPTD

SEQ ID NO: 33 - protein, 7 aa, synthetic construct
LPFPQPN

SEQ ID NO: 34 - protein, 7 aa, synthetic construct
RNVIGVD

SEQ ID NO: 35 - protein, 7 aa, synthetic construct
PSQGPID

SEQ ID NO: 36 - protein, 7 aa, synthetic construct
PASLPMT

SEQ ID NO: 37 - protein, 7 aa, synthetic construct
PPKPLMG

SEQ ID NO: 38 - protein, 7 aa, synthetic construct
RAHLPTS

SEQ ID NO: 39 - protein, 7 aa, synthetic construct
PALPSPY

SEQ ID NO: 40 - protein, 7 aa, synthetic construct
IPRSPSN

SEQ ID NO: 41 - protein, 7 aa, synthetic construct
TPKLPLN

SEQ ID NO: 42 - protein, 7 aa, synthetic construct
PTRPLEG

SEQ ID NO: 43 - protein, 7 aa, synthetic construct
QSPPPTT

SEQ ID NO: 44 - protein, 7 aa, synthetic construct
PAFPPGP

SEQ ID NO: 45 - protein, 7 aa, synthetic construct
VPRMPMS

SEQ ID NO: 46 - protein, 7 aa, synthetic construct
SVPPPRD

SEQ ID NO: 47 - protein, 7 aa, synthetic construct
PLRAPPL

SEQ ID NO: 48 - protein, 7 aa, synthetic construct
TPSTPFL

SEQ ID NO: 49 - protein, 7 aa, synthetic construct
KPNTPML

SEQ ID NO: 50 - protein, 7 aa, synthetic construct
PPRHPPA

SEQ ID NO: 51 - protein, 7 aa, synthetic construct
HPSFYMI

SEQ ID NO: 52 - protein, 7 aa, synthetic construct
THDRFCL

SEQ ID NO: 53 - protein, 7 aa, synthetic construct
MTLCTFK

SEQ ID NO: 54 - protein, 7 aa, synthetic construct
RVNVPVS

SEQ ID NO: 55 - protein, 7 aa, synthetic construct
PALPANL

SEQ ID NO: 56 - protein, 7 aa, synthetic construct
CLHPRTA

SEQ ID NO: 57 - protein, 7 aa, synthetic construct
DKFATRT

SEQ ID NO: 58 - protein, 7 aa, synthetic construct
PSVPLEG

SEQ ID NO: 59 - protein, 7 aa, synthetic construct
PASRPAM

SEQ ID NO: 60 - protein, 7 aa, synthetic construct
GRPPMNG

SEQ ID NO: 61 - protein, 7 aa, synthetic construct
CRAIPQR

SEQ ID NO: 62 - protein, 7 aa, synthetic construct
HKLPQPY

SEQ ID NO: 63 - protein, 7 aa, synthetic construct
AYPCPSR

SEQ ID NO: 64 - protein, 7 aa, synthetic construct
HTAPPAD

SEQ ID NO: 65 - protein, 7 aa, synthetic construct
ETRPVEQ

SEQ ID NO: 66 - skipped

SEQ ID NO: 67 - protein, 7 aa, synthetic construct
GPRTPTI

SEQ ID NO: 68 - protein, 7 aa, synthetic construct
NIACLAH